(19) United States
(12) Patent Application Publication
(10) Pub. No.: US 2026/0062445 A1
(43) Pub. Date: Mar. 5, 2026

(54) PEPTIDES WITH CYTOKINE-RELEASING ACTIVITY AND METHODS FOR CHARACTERIZING CYTOKINE-RELEASING PROFILES OF PEPTIDES

(71) Applicant: KOLIBER BIOSCIENCES INC., San Diego, CA (US)

(72) Inventors: Ewa LIS, San Diego, CA (US); Lily Kang LINDMEIER, San Diego, CA (US)

(21) Appl. No.: 19/108,220

(22) PCT Filed: Sep. 7, 2023
(86) PCT No.: PCT/US2023/073665

§ 371 Date: Mar. 3, 2025

Related U.S. Application Data
(60) Provisional application No. 63/405,169, filed on Sep. 9, 2022.

(51) Int. Cl. C07K 7/08; A61K 38/00; A61P 37/06; C07K 14/54; C12N 5/00; C12N 5/0783; G01N 33/50; G01N 33/543; G01N 33/68

(52) U.S. Cl. CPC C07K 7/08; A61P 37/06; C07K 14/5428; C12N 5/0018; C12N 5/0636; G01N 33/5023; G01N 33/505; G01N 33/543; G01N 33/6863; A61K 38/00; C12N 2501/2302; C12N 2501/51; C12N 2501/515; G01N 2333/5428; G01N 2333/57

(57) ABSTRACT

Disclosed herein are peptides that exhibit cytokine-releasing activity. The peptides are discovered through an artificial intelligence (AI) platform. Further disclosed herein are assays for characterizing the cytokine-releasing profiles of the peptides.

### CROSS-REFERENCE

The present application claims the benefit of U.S. Provisional Application No. 63/405,169 filed Sep. 9, 2022 which is incorporated herein by reference in its entirety.

### STATEMENT AS TO FEDERALLY SPONSORED RESEARCH

This invention was made with government support under Contract number 2014327 awarded by the National Science Foundation. The government has certain rights in the invention.

### SEQUENCE LISTING

The instant application contains a Sequence Listing which has been submitted electronically in XML format and is hereby incorporated by reference it its entirety. Said XML copy, created on Aug. 17, 2023, is named 63521-702_601SL.xml and is 39,987 bytes in size.

### BACKGROUND

Peptide therapeutics represent a key drug modality with unparalleled efficacy and safety in metabolic diseases and unique properties that are driving adoption of this modality in diverse therapeutic areas such as CNS, cardiovascular, and cancer therapies.

### SUMMARY

In an aspect, the present disclosure provides a peptide comprising an amino acid sequence at least 80% identical to an amino acid sequence selected from KLAKRLLLSMNQ (SEQ ID NO: 1) and FAVPAKQAKKTLT (SEQ ID NO: 3), or an amino acid sequence comprising LALLAPKLLRRF (SEQ ID NO: 2). In some embodiments, the peptide comprises an amino acid sequence selected from the group consisting of KLAKRLLLSMNQ (SEQ ID NO: 1), LALLAPKLLRRF (SEQ ID NO: 2), and FAVPAKQAKKTLT (SEQ ID NO: 3).

In some embodiments, the peptide comprises the amino acid sequence KLAKRLLLSMNQ (SEQ ID NO: 1). In some embodiments, the peptide consists of the amino acid sequence KLAKRLLLSMNQ (SEQ ID NO: 1). In some embodiments, the peptide comprises the amino acid sequence LALLAPKLLRRF (SEQ ID NO: 2). In some embodiments, the peptide consists of the amino acid sequence LALLAPKLLRRF (SEQ ID NO: 2). In some embodiments, the peptide comprises the amino acid sequence FAVPAKQAKKTLT (SEQ ID NO: 3). In some embodiments, the peptide consists of the amino acid sequence

(SEQ ID NO: 3)
FAVPAKQAKKTLT.

In some embodiments, the peptide elicits release of at least one anti-inflammatory cytokine from an immune cell. In some embodiments, the at least one anti-inflammatory cytokine is selected from interleukin 10 (IL-10), interleukin 4 (IL-4), interleukin 13 (IL-13), interleukin 22 (IL-22), interleukin 6 (IL-6), and transforming growth factor beta (TGF-β). In some embodiments, the at least one anti-inflammatory cytokine is interleukin 10 (IL-10). In some embodiments, the immune cell is a CD4 positive T cell. In some embodiments, the immune cell is a human CD4+ T cell. In some embodiments, the peptide is a therapeutic peptide for treatment of an inflammatory disease. In some embodiments, the peptide is a therapeutic peptide for treatment of an allergy or an allergic disease.

In an aspect, the present disclosure provides an anti-inflammatory peptide (AIP) comprising an amino acid sequence of KLAKRLLLSMNQ (SEQ ID NO: 1), LALLAPKLLRRF (SEQ ID NO: 2), FAVPAKQAKKTLT (SEQ ID NO: 3), or an amino acid sequence that has 1, 2, or 3 amino acid substitutions, additions, or deletions relative to the amino acid sequences of KLAKRLLLSMNQ (SEQ ID NO: 1), LALLAPKLLRRF (SEQ ID NO: 2), or FAVPAKQAKKTLT (SEQ ID NO: 3), wherein the AIP consists of 10-16 amino acid residues.

In some embodiments, the AIP comprises an amino acid sequence of KLAKRLLLSMNQ (SEQ ID NO: 1), LALLAPKLLRRF (SEQ ID NO: 2), FAVPAKQAKKTLT (SEQ ID NO: 3), or an amino acid sequence that has at most 1 amino acid substitution, addition, or deletion relative to the amino acid sequence of KLAKRLLLSMNQ (SEQ ID NO: 1), LALLAPKLLRRF (SEQ ID NO: 2), or FAVPAKQAKKTLT (SEQ ID NO: 3).

In some embodiments, the AIP comprises an amino acid sequence of KLAKRLLLSMNQ (SEQ ID NO: 1), LALLAPKLLRRF (SEQ ID NO: 2), or FAVPAKQAKKTLT (SEQ ID NO: 3). In some embodiments, the AIP comprises the amino acid sequence of KLAKRLLLSMNQ (SEQ ID NO: 1). In some embodiments, the AUp consists of the amino acid sequence of KLAKRLLLSMNQ (SEQ ID NO: 1). In some embodiments, the AIP comprises the amino acid sequence of LALLAPKLLRRF (SEQ ID NO: 2). In some embodiments, the AIP consists of the amino acid sequence of LALLAPKLLRRF (SEQ ID NO: 2). In some embodiments, the AIP comprises the amino acid sequence of FAVPAKQAKKTLT (SEQ ID NO: 3). In some embodiments, the AIP consists of the amino acid sequence of

(SEQ ID NO: 3)
FAVPAKQAKKTLT.

In some embodiments, the AIP elicits release of at least one anti-inflammatory cytokine from a CD4 positive T cell. In some embodiments, the at least one anti-inflammatory cytokine is interleukin 10 (IL-10). In some embodiments, the at least one anti-inflammatory cytokine is selected from interleukin 10 (IL-10), interleukin 4 (IL-4), interleukin 13 (IL-13), interleukin 22 (IL-22), interleukin 6 (IL-6), and transforming growth factor beta (TGF-β). In some embodiments, the AIP is a therapeutic peptide for treatment of an inflammatory disease. In some embodiments, the AIP is a therapeutic peptide for treatment of an allergy or an allergic disease.

In an aspect, the present disclosure provides an anti-inflammatory peptide (AIP) comprising an amino acid sequence of Formula I.

(Formula I)
X₁-A-X₃-X₄-X₅-X₆-X₇-X₈-K-X₁₀-T-X₁₂-T,

wherein X₁ is F or H; X₃ is V, Y, W, or F; X₄ is P, Y, W, V, T, R, M, K, I, H, F, or E; X₅ is A or W; X₆ is K, Y, W, M, L, I, or F; X₇ is Q or M; X₈ is A or N; X₁₀ is K, W, F, or C; and X₁₂ is L, T, Q, E, or A.

In some embodiments of Formula I, X₁ is F. In some embodiments of Formula I, X₃ is V. In some embodiments of Formula I, X₄ is selected from P, W, V, M, I, and F. In some embodiments of Formula I, X₄ is P. In some embodiments of Formula I, X₅ is A. In some embodiments of Formula I, X₆ is selected from K, Y, W, and F. In some embodiments of Formula I, X₆ is K. In some embodiments of Formula I, X₇ is Q. In some embodiments of Formula I, X₈ is A. In some embodiments of Formula I, X₁₀ is K, W, or C. In some embodiments of Formula I, X₁₀ is K. In some embodiments of Formula I, X₁₂ is L or E. In some embodiments, X₁₂ is L. In some embodiments of Formula I, the AlP comprises an amino acid sequence according to any one of SEQ ID NOs 3-26.

In some embodiments, the AIP elicits release of at least one anti-inflammatory cytokine from an immune cell. In some embodiments, the at least one anti-inflammatory cytokine is selected from interleukin 10 (IL-10), interleukin 4 (IL-4), interleukin 13 (IL-13), interleukin 22 (IL-22), interleukin 6 (IL-6), and transforming growth factor beta (TGF-β). In some embodiments, the at least one anti-inflammatory cytokine is interleukin 10 (IL-10). In some embodiments, the immune cell is a CD4 positive T cell. In some embodiments, the immune cell is a human CD4 positive T cell. In some embodiments, the AlP is a therapeutic peptide for treatment of an inflammatory disease. In some embodiments, the AIP is a therapeutic peptide for treatment of an allergy or an allergic disease.

In an aspect, the present disclosure provides an anti-inflammatory peptide (AIP) comprising an amino acid sequence of Formula II:

(Formula II)
L-X₂-X₃-X₄-X₅-X₆-K-L-X₉-X₁₀-X₁₁-X₁₂,

wherein X₂ is A, Y, W, V, R, Q, M, L, K, I, H, or F; X₃ is L, W, or F; X₄ is L, W, N, or C; X₅ is A, W, or F; X₆ is P, Y, W, V, T, S, R, Q, N, M, L, I, H, G, F, D, C, or A; X₉ is L or W; X₁₀ is R or S; X₁₁ is R or Q; and X₁₂ is F, V, T, S, R, Q, P, N, K, I, G, E, D, C, or A.

In some embodiments of Formula II, X₂ is A, Y, W, or F. In some embodiments of Formula II, X₂ is A. In some embodiments of Formula II, X₃ is L or W. In some embodiments of Formula II, X₃ is L. In some embodiments of Formula II, X₄ is L or W. In some embodiments of Formula II, X₄ is L. In some embodiments of Formula II, X₅ is A. In some embodiments of Formula II, X₆ is P, W, M, H, F, or C. In some embodiments of Formula II, X₆ is P. In some embodiments of Formula II, X₉ is L. In some embodiments of Formula II, X₁₀ is R. In some embodiments of Formula II, X₁₁ is R. In some embodiments of Formula II, X₁₂ is F, P, K, G, E, D, C, or A. In some embodiments of Formula II, X₁₂ is F, K, E, D, or A. In some embodiments of Formula II, X₁₂ is F. In some embodiments of Formula II, the AlP comprises an amino acid sequence selected from any one of SEQ ID NOs 2 and 27-45.

In some embodiments, the AIP elicits release of at least one anti-inflammatory cytokine from an immune cell. In some embodiments, the at least one anti-inflammatory cytokine is selected from interleukin 10 (IL-10), interleukin 4 (IL-4), interleukin 13 (IL-13), interleukin 22 (IL-22), interleukin 6 (IL-6), and transforming growth factor beta (TGF-β). In some embodiments, the at least one anti-inflammatory cytokine is interleukin 10 (IL-10). In some embodiments, the immune cell is a CD4 positive T cell. In some embodiments, the immune cell is a human CD4 positive T cell. In some embodiments, the AlP is a therapeutic peptide for treatment of an inflammatory disease. In some embodiments, the AlP is a therapeutic peptide for treatment of an allergy or an allergic disease.

In an aspect, the present disclosure provides methods for characterizing a cytokine profile induced by a peptide comprising: (a) isolating naïve CD4 positive T cells from peripheral blood mononuclear cells (PBMCs); (b) expanding the isolated naïve CD4 positive T cells; (c) co-culturing the expanded naïve CD4 positive T cells with PBMCs and stimulating the expanded naïve CD4 positive T cells one or more times with the peptide; and (d) detecting a signal corresponding to a level of one or more cytokines produced by the expanded naïve CD4 positive T cells.

In some embodiments, expanding the isolated naïve CD4 positive T cells leads to at least a 20-fold expansion of the isolated naïve CD4 positive T cells. In some embodiments, expanding the isolated naïve CD4 positive T cells leads to at least a 30-fold expansion of the isolated naïve CD4 positive T cells.

In some embodiments, the stimulating is performed in the presence of at least one helper cytokine. In some embodiments, the at least one helper cytokine is selected from interleukin-2 (IL-2) and interleukin-7 (IL-7). In some embodiments, the at least one helper cytokine is IL-2.

In some embodiments, the stimulating is performed at least two times. In some embodiments, the stimulating is performed at least three times. In some embodiments, the stimulating is performed at least four times. In some embodiments, each of the stimulating steps is performed for a time period of about 1 day to about 3 days. In some embodiments, each of the stimulating steps is performed for a time period of about 3 days. In some embodiments, each of the stimulating steps comprises suspending the expanded naïve CD4 positive T cells in a fresh volume of media and stimulating the expanded naïve CD4 positive T cells with a fresh sample of the peptide. In some embodiments, each of the stimulating steps is performed in the presence of about 1 μg/mL to about 100 μg/mL of the peptide. In some embodiments, each of the stimulating steps is performed in the presence of about 10 μg/mL to about 50 μg/ml of the peptide. In some embodiments, each of the stimulating steps is performed in the presence of about 40 μg/mL of the peptide.

In some embodiments, the co-culturing is performed with an excess of the expanded naïve CD4 positive T cells relative to the PBMCs. In some embodiments, a ratio of the expanded naïve CD4 positive T cells to the PBMCs during the co-culturing ranges from about 2:1 to about 50:1. In some embodiments, a ratio of the expanded naïve CD4 positive T cells to the PBMCs during the co-culturing ranges from about 1:1 to about 25:1. In some embodiments, a ratio of the expanded naïve CD4 positive T cells to the PBMCs during the co-culturing ranges from about 1:1 to about 10:1. In some embodiments, a ratio of the expanded naïve CD4 positive T cells to the PBMCs during the co-culturing ranges from about 2:1 to about 8:1. In some embodiments, a ratio of the expanded naïve CD4 positive T cells to the PBMCs during the co-culturing ranges from about 2:1 to about 50:1. In some embodiments, a ratio of the expanded naïve CD4 positive T cells to the PBMCs during the co-culturing is about 4:1.

In some embodiments, expanding the isolated naïve CD4 positive T cells comprises exposing the isolated naïve CD4 positive T cells to magnetic beads coated with anti-CD3 antibodies and anti-CD28 antibodies.

In some embodiments, detecting the signal corresponding to the level of the one or more cytokines comprises detecting the signal by an enzyme-linked immunoassay (ELISA). In some embodiments, detecting the signal corresponding to the level of the one or more cytokines comprises detecting the signal by an enzyme-linked immunosorbent spot (ELISpot). In some embodiments, detecting the signal corresponding to the level of the one or more cytokines comprises detecting the signal by a flow cytometry-based assay.

In some embodiments, the one or more cytokines include one or more anti-inflammatory cytokines. In some embodiments, the one or more cytokines include one or more pro-inflammatory cytokines. In some embodiments, the one or more anti-inflammatory cytokines are selected from interleukin 10 (IL-10), interleukin 4 (IL-4), interleukin 13 (IL-13), interleukin 22 (IL-22), interleukin 6 (IL-6), and transforming growth factor beta (TGF-β). In some embodiments, the one or more anti-inflammatory cytokines include IL-10. In some embodiments, the one or more pro-inflammatory cytokines include interferon gamma (IFN-γ). In some embodiments, the signal corresponding to a level of one or more cytokines is detected in a media or in a cell.

In an aspect, the present disclosure provides a method of eliciting a release of one or more anti-inflammatory cytokines from an immune cell comprising contacting the immune cell with a peptide or an AIP of any one of the preceding embodiments.

In an aspect, the present disclosure provides a method of using a peptide or an AlP of any one of the preceding embodiments as a positive control for eliciting release of at least one anti-inflammatory cytokine from a cell in an immune stimulating assay. In some embodiments, the anti-inflammatory cytokine is selected from interleukin 10 (IL-10), interleukin 4 (IL-4), interleukin 13 (IL-13), interleukin 22 (IL-22), interleukin 6 (IL-6), transforming growth factor beta (TGF-$), and combinations thereof. In some embodiments, the cell is an immune cell. In some embodiments, the cell is a T cell. In some embodiments, the cell is a CD4 positive T cell.

In an aspect, the present disclosure provides a method of treating or preventing a disease or disorder in a subject in need thereof, wherein the method comprises administering a peptide or an AIP of any one of the preceding embodiments to the subject. In some embodiments, the disease or disorder is an inflammatory disease. In some embodiments, the disease or disorder is an allergy or an allergic disease.

In an aspect, the present disclosure provides pharmaceutical compositions for treating or preventing a disease or disorder in a subject. In some embodiments, a pharmaceutical composition of the present disclosure comprises a peptide or an AIP of any one of the preceding embodiments. In some embodiments, the pharmaceutical composition further comprises one or more pharmaceutically acceptable excipients. In some embodiments, the disease or disorder is an inflammatory disease. In some embodiments, the disease or disorder is an allergy or an allergic disease.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Some of the novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-IF illustrate levels for interleukin 10 (IL-10) and interferon-gamma (IFN-γ) in individual wells after each of three peptide stimulations as measured using a peripheral blood mononuclear cell (PBMC) assay to test activation of T cells;

FIG. 2 illustrates an expanded CD4 positive T cell assay in accordance with the present disclosure;

FIGS. 3A-3D illustrate cytokine levels (IL-10 and IFN-γ) in individual wells after each of two peptide stimulations as measured using the expanded CD4 positive T cell assay as described herein;

FIG. 4 shows a comparison of IL-10 signal and well-to-well variation after two peptide stimulations using the expanded CD4 positive T cell assay (CD4 expansion) and the PBMC assay (PBMC only) as described herein;

FIGS. 5A-5D show the fraction of responding wells for IL-10 and IFN-γ after first and second peptide stimulations as measured using the expanded CD4 positive T cell assay as described herein;

FIGS. 6A-6B show a comparison of the fraction of responding wells for IL-10 between the PBMC assay (FIG. 6A) and the expanded CD4 positive T cell assay (FIG. 6B) as described herein;

FIGS. 7A-7B show the fraction of responding wells for IL-10 (FIG. 7A) and IFN-γ (FIG. 7B) after peptide stimulations across different donors expressing the HLA-DRB1*1101 allele;

FIG. 8 shows a heatmap of substitution predictions for peptide KBP82 (SEQ ID NO: 3) generated using a machine learning model, whereby decreases in probability for anti-inflammatory activity have more blue hue in the color scale and increases in probability for anti-inflammatory activity have more red hue in the color scale; and

FIG. 9 shows a heatmap of substitution predictions for peptide KBP73 (SEQ ID NO: 2) generated using a machine learning model, whereby decreases in probability for anti-inflammatory activity have more blue hue in the color scale and increases in probability for anti-inflammatory activity have more red hue in the color scale.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Unless defined otherwise, all terms of art, notations and other technical and scientific terms or terminology used herein are intended to have the same meaning as is commonly understood by one of ordinary skill in the art to which the claimed subject matter pertains. In some embodiments, terms with commonly understood meanings are defined herein for clarity and/or for ready reference, and the inclusion of such definitions herein should not necessarily be construed to represent a substantial difference over what is generally understood in the art.

### Definitions

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, e.g., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The terms “determining,” “measuring,” “evaluating,” “assessing,” “assaying,” and “analyzing” may be used interchangeably herein to refer to forms of measurement. The terms include determining if an element is present or not (for example, detection). These terms can include quantitative, qualitative or quantitative, and qualitative determinations. Assessing can be relative or absolute.

The term “subject,” “patient,” or “individual” as used herein can encompass a mammal and a non-mammal. A mammal can be any member of the mammalian class, including but not limited to a human, a non-human primates such as a chimpanzee, an ape or other monkey species; a farm animal such as cattle, a horse, a sheep, a goat, a swine; a domestic animal such as a rabbit, a dog (or a canine), and a cat (or a feline); a laboratory animal including a rodent, such as a rat, a mouse and a guinea pig, and the like. A non-mammal can include a bird, a fish and the like. In some embodiments, a subject can be a mammal. In some embodiments, a subject can be a human. In some instances, a human can be an adult. In some instances, a human can be a child. In some instances, a human can be age 0-17 years old. In some instances, a human can be age 18-130 years old. In some instances, a subject can be diagnosed with, or can be suspected of having, a condition or disease. A subject can be a patient. A subject can be an individual. In some instances, a subject, patient or individual can be used interchangeably.

The terms “treat,” “treating,” “treatment,” “ameliorate,” or “ameliorating” and other grammatical equivalents as used herein, generally can include alleviating, or abating a disease or condition symptoms, inhibiting a disease or condition, e.g., arresting the development of a disease or condition, relieving a disease or condition, causing regression of a disease or condition, relieving a condition caused by the disease or condition, or stopping symptoms of a disease or condition. The term “preventing” generally refers to ameliorating or preventing the underlying causes of symptoms, and can include prophylaxis.

The term “peptide” generally refers to a polymer of at least two amino acid residues, wherein the amino acids are joined by peptide bonds. This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The term includes amino acid chains of any length, including full length proteins, and proteins with or without secondary and/or tertiary structure (e.g., domains). The term also encompasses an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include non-natural amino acids which have been chemically modified to include a group or a chemical moiety not naturally present on the amino acid. Amino acid analogues may refer to amino acid derivatives. The term “amino acid” includes both D-amino acids and L-amino acids.

In some cases, amino acids can be canonical amino acids such as the 20 proteinogenic L-amino acids. In some cases, amino acids can be unnatural amino acids. An “unnatural amino acid” as described herein can include any amino acid other than one of the 20 proteinogenic proteins in an L-configuration. Such amino acids can include amino acids with non-canonical side chains, D-amino acids, β-amino acids, and the like. The peptides as disclosed herein may be chemically synthesized, produced using a recombinant expression system, or via a combination of chemical and recombinant means.

A peptide described herein can be useful as an anti-inflammatory peptide. The term “anti-inflammatory peptide” generally refers to a peptide that exhibits an anti-inflammatory property or elicits an anti-inflammatory response. In some cases, an anti-inflammatory response can comprise a release of cytokines that are generally considered to be anti-inflammatory, such as IL-10, from a cell. In some cases, an “anti-inflammatory peptide” as used herein is a peptide that elicits release of one or more anti-inflammatory cytokines from an immune cell, such as a T cell, when the immune cell is contacted with the peptide. An “anti-inflammatory cytokine” is a cytokine capable of reducing or preventing inflammation in a subject. Thus, an “anti-inflammatory peptide” as used herein may be a peptide that is capable of reducing or inhibiting inflammation in a subject treated with the peptide.

As used herein, “anti-inflammatory activity” refers to a property of a peptide, whereby the peptide is capable of eliciting release of one or more anti-inflammatory cytokines from an immune cell when the immune cell is contacted with the peptide. In some cases, “anti-inflammatory activity” refers to a property of a peptide, whereby the peptide has the ability to reduce or inhibit inflammation in a subject treated with the peptide.

“Predicted probability for anti-inflammatory activity” as used herein is a probability between 0 and 1 that a peptide is an anti-inflammatory peptide (AIP) which is output by a machine learning model of the present disclosure.

The term “cytokine” generally refers to small soluble proteins secreted by cells that can alter the behavior or properties of the secreting cell or another cell. Cytokines bind to cytokine receptors and trigger a behavior or property within the cell, for example, cell proliferation, death or differentiation. Example cytokines include, but are not limited to, interleukins (e.g., IL-2, IL-3, IL-4, IL-5, IL-6, IL-7, IL-8, IL-9, IL-10, IL-11, IL-12, IL-13, IL-15, IL-16, IL-17, IL-18, IL-1α, IL-β, and IL-1 RA), granulocyte colony stimulating factor (G-CSF), granulocyte-macrophage colony stimulating factor (GM-CSF), oncostatin M, erythropoietin, leukemia inhibitory factor (LIF), interferons, B7.1 (also known as CD80), B7.2 (also known as B70, CD86), TNF family members (TNF-α, TNF-β, LT-0, CD40 ligand, Fas ligand, CD27 ligand, CD30 ligand, 4-1BBL, Trail), and MIF.

A “naïve T cell” generally refers to a T cell that has matured but has not yet encountered its corresponding antigen. A “CD4 positive T cell” refers to a T helper cell that expresses the surface protein CD4 and aids in the activity of other immune cells by releasing cytokines.

“Stimulating” with a peptide as used herein refers to contacting T cells (e.g., naïve CD4 positive T cells) with a peptide or exposing the T cells to a peptide under conditions that permit interaction of the T cell receptors with the peptide-MHC complex presented on antigen-presenting cells, resulting in release of cytokines from the T cells.

“Expanding” or “expansion” of T cells as disclosed herein refers to polyclonal expansion of T cells whereby T cells are activated and proliferated. In some non-limiting embodiments, the expansion of T cells as disclosed herein may be carried out by exposing T cells to beads coated with anti-CD3 and anti-CD28 to stimulate T cells in a manner that partially mimics stimulation by antigen-presenting cells.

As used herein, the term “therapeutically effective amount” refers to an amount of a peptide or other therapeutic agent effective to deliver a therapeutic benefit to a subject to whom the peptide or other therapeutic agent is administered. For example, a “therapeutically effective amount” may be an amount of a peptide or other therapeutic agent effective to relieve, reduce, abate, prevent, or eradicate one or more symptoms associated with a disease or disorder of a subject to whom the peptide or therapeutic agent is administered.

The terms “co-administration” or “administering in combination with,” and their grammatical equivalents or the like, as used herein, generally encompass administration of selected therapeutic agents to a subject and can include treatment regimens in which agents are administered by the same or different routes of administration or at the same or different times. In some embodiments, a peptide disclosed herein can be co-administered with other agents. These terms can encompass administration of two or more agents to a subject so that both agents and/or their metabolites are present in the subject at the same time. They can include simultaneous administration, administration at different times, and/or administration in a composition in which both agents are present. Thus, in some embodiments, a peptide and an additional agent(s) can be administered in a single composition. In some embodiments, a peptide and an additional agent(s) can be admixed in the composition. In some embodiments, a same peptide or agent can be administered via a combination of different routes of administration. In some embodiments, each agent can be administered in a therapeutically effective amount.

The term “pharmaceutically acceptable carrier” as used herein includes, but is not limited to, any carrier that does not interfere with the effectiveness of the biological activity of the ingredients of a pharmaceutical composition and is not toxic to the subject or patient to whom it is administered. Examples of suitable pharmaceutical carriers may include, but are not limited to, phosphate buffered saline solutions, water, emulsions, such as oil/water emulsions, various types of wetting agents, sterile solutions etc.

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The Smith Waterman algorithm can also be used to determine percent identity. Exemplary parameters for amino acid sequence comparison include the following: 1) algorithm from Needleman and Wunsch (J. Mol. Biol., 48:443-453 (1970)); 2) BLOSSUM62 comparison matrix from Hentikoff and Hentikoff (Proc. Nat. Acad. Sci. USA., 89:10915-10919 (1992)) 3) gap penalty=12; and 4) gap length penalty=4. A program useful with these parameters can be publicly available as the “gap” program (Genetics Computer Group, Madison, Wis.). The aforementioned parameters are the default parameters for polypeptide comparisons (with no penalty for end gaps). Alternatively, polypeptide sequence identity can be calculated using the following equation: % identity−(the number of identical residues)/(alignment length in amino acid residues)*100. For this calculation, alignment length includes internal gaps but does not include terminal gaps.

As used herein, an “immune stimulating assay” refers to a method of measuring an immune response to a stimulus where the measurement of an immune response is based on quantifying an effect on the immune cells (for example, the release of cytokines from immune cells) following contact of the immune cells with an antigen such as a peptide, protein, or protein fragment (the stimulus).

Peptides with Cytokine Releasing Activity

Development of peptide therapeutics is slow compared to antibodies and costly compared to small molecules. The development process requires not only discovery of high affinity molecules from high throughput screens but also attainment of multiple performance metrics such as activity, selectivity, solubility, stability and sometimes oral availability. Current methods of analyzing complex datasets in spreadsheets are no longer appropriate for rapid development. Moreover, existing computational tools are underdeveloped technically, commercially making them unsuitable for use by wet lab scientists.

To overcome these and other challenges, described herein are novel peptides discovered through an artificial intelligence (AI)-based approach to peptide drug discovery as disclosed in U.S. patent application No. 63/354,006 filed on Jun. 21, 2022 and incorporated herein by reference. The peptides are predicted to elicit anti-inflammatory responses by inducing release of anti-inflammatory cytokines in human immune cells, and may serve as leads for further development of peptide therapeutics for inflammatory diseases. The AI platform utilizes deep neural networks and peptide encodings to train machine learning models which are then applied to identify hits or predict variants with improved performance and accelerate peptide development projects. Novel assays for wet lab validation of the cytokine releasing profiles of the predicted peptides are further described herein.

In an aspect, the present disclosure provides a peptide comprising an amino acid sequence at least 80% identical to an amino acid sequence selected from KLAKRLLLSMNQ (SEQ ID NO: 1) and FAVPAKQAKKTLT (SEQ ID NO: 3), or an amino acid sequence comprising LALLAPKLLRRF (SEQ ID NO: 2) (see Table 1).

TABLE 1
Peptide Sequences with Anti-inflammatory Activity

| Peptide | Sequence | SEQ ID NO. |
|---|---|---|
| KBP63 | KLAKRLLLSMNQ | 1 |
| KBP73 | LALLAPKLLRRF | 2 |
| KBP82 | FAVPAKQAKKTLT | 3 |

In some embodiments, the peptide comprises an amino acid sequence at least 70% identical to the amino acid sequence KLAKRLLLSMNQ (SEQ ID NO: 1). In some embodiments, the peptide comprises an amino acid sequence at least 75% identical to the amino acid sequence KLAKRLLLSMNQ (SEQ ID NO: 1). In some embodiments, the peptide comprises an amino acid sequence at least 80% identical to the amino acid sequence KLAKRLLLSMNQ (SEQ ID NO: 1). In some embodiments, the peptide comprises an amino acid sequence at least 85% identical to the amino acid sequence KLAKRLLLSMNQ (SEQ ID NO: 1). In some embodiments, the peptide comprises an amino acid sequence that is at least 90% identical to the amino acid sequence KLAKRLLLSMNQ (SEQ ID NO: 1). In some embodiments, the peptide comprises an amino acid sequence that is at least 92% identical to the amino acid sequence KLAKRLLLSMNQ (SEQ ID NO: 1). In some embodiments, the peptide comprises an amino acid sequence that is at least 95% identical to the amino acid sequence KLAKRLLLSMNQ (SEQ ID NO: 1). In some embodiments, the peptide comprises an amino acid sequence that is at least 99% identical to the amino acid sequence KLAKRLLLSMNQ (SEQ ID NO: 1).

In some embodiments, the peptide comprises an amino acid sequence that is at least 70% identical to the amino acid sequence FAVPAKQAKKTLT (SEQ ID NO: 3). In some embodiments, the peptide comprises an amino acid sequence that is at least 75% identical to the amino acid sequence FAVPAKQAKKTLT (SEQ ID NO: 3). In some embodiments, the peptide comprises an amino acid sequence that is at least 80% identical to the amino acid sequence FAVPAKQAKKTLT (SEQ ID NO: 3). In some embodiments, the peptide comprises an amino acid sequence that is at least 85% identical to the amino acid sequence FAVPAKQAKKTLT (SEQ ID NO: 3). In some embodiments, the peptide comprises an amino acid sequence that is at least 90% identical to the amino acid sequence FAVPAKQAKKTLT (SEQ ID NO: 3). In some embodiments, the peptide comprises an amino acid sequence that is at least 92% identical to the amino acid sequence FAVPAKQAKKTLT (SEQ ID NO: 3). In some embodiments, the peptide comprises an amino acid sequence that is at least 95% identical to the amino acid sequence FAVPAKQAKKTLT (SEQ ID NO: 3). In some embodiments, the peptide comprises an amino acid sequence that is at least 99% identical to the amino acid sequence FAVPAKQAKKTLT (SEQ ID NO: 3).

In some embodiments, the peptide comprises an amino acid sequence selected from the group consisting of KLAKRLLLSMNQ (SEQ ID NO: 1), LALLAPKLLRRF (SEQ ID NO: 2), and FAVPAKQAKKTLT (SEQ ID NO: 3). In some embodiments, the peptide comprises the amino acid sequence KLAKRLLLSMNQ (SEQ ID NO: 1). In some embodiments, the peptide consists of the amino acid sequence KLAKRLLLSMNQ (SEQ ID NO: 1). In some embodiments, the peptide comprises the amino acid sequence LALLAPKLLRRF (SEQ ID NO: 2). In some embodiments, the peptide consists of the amino acid sequence LALLAPKLLRRF (SEQ ID NO: 2). In some embodiments, the peptide comprises the amino acid sequence FAVPAKQAKKTLT (SEQ ID NO: 3). In some embodiments, the peptide consists of the amino acid sequence FAVPAKQAKKTLT (SEQ ID NO: 3).

In some embodiments, the peptide elicits release of at least one anti-inflammatory cytokine from an immune cell. In some embodiments, the at least one anti-inflammatory cytokine is selected from interleukin 10 (IL-10), interleukin 4 (IL-4), interleukin 13 (IL-13), interleukin 22 (IL-22), interleukin 6 (IL-6), and transforming growth factor beta (TGF-β). In some embodiments, the at least one anti-inflammatory cytokine is IL-10. In some embodiments, the at least one anti-inflammatory cytokine is IL-4. In some embodiments, the anti-inflammatory cytokine is IL-13. In some embodiments, the anti-inflammatory cytokine is IL-22.

In some embodiments, the anti-inflammatory cytokine is IL-6. In some embodiments, the anti-inflammatory cytokine is TGF-β. In some embodiments, the peptide elicits release of two or more anti-inflammatory cytokines from an immune cell. In some embodiments, the two or more anti-inflammatory cytokines are selected from IL-10, IL-4, IL-13, IL-22, IL-6, and TGF-β. In some embodiments, the peptide elicits release of at least one pro-inflammatory cytokine from an immune cell. In some embodiments, the at least one pro-inflammatory cytokine includes interferon gamma (IFN-γ). In some embodiments, the immune cell is a T cell. In some embodiments, the immune cell is a CD4 positive T cell. In some embodiments, the immune cell is a human CD4 positive T cell.

In some embodiments, the peptide described herein is an anti-inflammatory peptide (AIP). In some embodiments, the peptide is a therapeutic peptide for treatment of a disease or disorder. In some embodiments, the peptide is a therapeutic peptide for treatment of an inflammatory disease. In some embodiments, the peptide is a therapeutic peptide for treatment of an allergy or allergic disease.

In an aspect, the present disclosure provides an anti-inflammatory peptide (AIP) comprising an amino acid sequence of KLAKRLLLSMNQ (SEQ ID NO: 1), LALLAPKLLRRF (SEQ ID NO: 2), FAVPAKQAKKTLT (SEQ ID NO: 3), or an amino acid sequence that has 1, 2, or 3 amino acid substitutions, additions, or deletions relative to the amino acid sequences of KLAKRLLLSMNQ (SEQ ID NO: 1), LALLAPKLLRRF (SEQ ID NO: 2), or FAVPAKQAKKTLT (SEQ ID NO: 3), wherein the AIP consists of 10-16 amino acid residues.

In some embodiments, the AIP consists of 12 to 16 amino acid residues. In some embodiments, the AIP consists of 12 to 14 amino acid residues. In some embodiments, the AIP consists of 12 to 13 amino acids. In some embodiments, the AIP comprises an amino acid sequence of KLAKRLLLSMNQ (SEQ ID NO: 1), LALLAPKLLRRF (SEQ ID NO: 2), FAVPAKQAKKTLT (SEQ ID NO: 3), or an amino acid sequence that has at most 1 amino acid substitution, addition, or deletion relative to the amino acid sequence of KLAKRLLLSMNQ (SEQ ID NO: 1), LALLAPKLLRRF (SEQ ID NO: 2), or FAVPAKQAKKTLT (SEQ ID NO: 3).

In some embodiments, the AlP comprises an amino acid sequence of KLAKRLLLSMNQ (SEQ ID NO: 1), LALLAPKLLRRF (SEQ ID NO: 2), or FAVPAKQAKKTLT (SEQ ID NO: 3). In some embodiments, the AIP comprises the amino acid sequence of KLAKRLLLSMNQ (SEQ ID NO: 1). In some embodiments, the AIP consists of the amino acid sequence of KLAKRLLLSMNQ (SEQ ID NO: 1). In some embodiments, the AIP comprises the amino acid sequence of LALLAPKLLRRF (SEQ ID NO: 2). In some embodiments, the AIP consists of the amino acid sequence of LALLAPKLLRRF (SEQ ID NO: 2). In some embodiments, the AIP comprises the amino acid sequence of FAVPAKQAKKTLT (SEQ ID NO: 3). In some embodiments, the AIP consists of the amino acid sequence of

(SEQ ID NO: 3)
FAVPAKQAKKTLT.

In some embodiments, the peptide elicits release of at least one anti-inflammatory cytokine from an immune cell. In some embodiments, the at least one anti-inflammatory cytokine is selected from interleukin 10 (IL-10), interleukin 4 (IL-4), interleukin 13 (IL-13), interleukin 22 (IL-22), interleukin 6 (IL-6), and transforming growth factor beta (TGF-β). In some embodiments, the at least one anti-inflammatory cytokine is IL-10. In some embodiments, the at least one anti-inflammatory cytokine is IL-4. In some embodiments, the anti-inflammatory cytokine is IL-13. In some embodiments, the anti-inflammatory cytokine is IL-22. In some embodiments, the anti-inflammatory cytokine is IL-6. In some embodiments, the anti-inflammatory cytokine is TGF-β. In some embodiments, the peptide elicits release of two or more anti-inflammatory cytokines from an immune cell. In some embodiments, the two or more anti-inflammatory cytokines are selected from IL-10, IL-4, IL-13, IL-22, IL-6, and TGF-β. In some embodiments, the peptide elicits release of at least one pro-inflammatory cytokine from an immune cell. In some embodiments, the at least one pro-inflammatory cytokine includes interferon gamma (IFN-γ). In some embodiments, the immune cell is a T cell. In some embodiments, the immune cell is a CD4 positive T cell. In some embodiments, the immune cell is a human CD4 positive T cell.

In some embodiments, the peptide is a therapeutic peptide for treatment of a disease or disorder. In some embodiments, the peptide is a therapeutic peptide for treatment of an inflammatory disease. In some embodiments, the peptide is a therapeutic peptide for treatment of an allergy or allergic disease. In some embodiments, the disease or disorder is associated with one or more anti-inflammatory cytokines. In some embodiments, the disease or disorder is associated with the deficiency of the one or more anti-inflammatory cytokines.

In an aspect, the present disclosure provides an anti-inflammatory peptide (AIP) comprising an amino acid sequence of Formula I.

(Formula I)
X₁-A-X₃-X₄-X₅-X₆-X₇-X₈-K-X₁₀-T-X₁₂-T,

wherein X₁ is F or H; X₃ is V, Y, W, or F; X₄ is P, Y, W, V, T, R, M, K, I, H, F, or E; X₅ is A or W; X₆ is K, Y, W, M, L, I, or F; X₇ is Q or M; X₈ is A or N; X₁₀ is K, W, F, or C; and X₁₂ is L, T, Q, E, or A.

In some embodiments of Formula I, X₁ is F. In some embodiments of Formula I, X₁ is H. In some embodiments of Formula I, X₃ is V. In some embodiments of Formula I, X₃ is Y. In some embodiments of Formula I, X₃ is W. In some embodiments of Formula I, X₃ is F. In some embodiments of Formula I, X₄ is selected from P, W, V, M, I, and F. In some embodiments of Formula I, X₄ is P. In some embodiments of Formula I, X₄ is Y. In some embodiments of Formula I, X₄ is W. In some embodiments of Formula I, X₄ is V. In some embodiments of Formula I, X₄ is T. In some embodiments of Formula I, X₄ is R. In some embodiments of Formula I, X₄ is M. In some embodiments of Formula I, X₄ is K. In some embodiments of Formula I, X₄ is I. In some embodiments of Formula I, X₄ is H. In some embodiments of Formula I, X₄ is F. In some embodiments of Formula I, X₄ is E.

In some embodiments of Formula I, X₅ is A. In some embodiments of Formula I, X₅ is W. In some embodiments of Formula I, X₆ is selected from K, Y, W, and F. In some embodiments of Formula I, X₆ is K. In some embodiments of Formula I, X₆ is Y. In some embodiments of Formula I, X₆ is W. In some embodiments of Formula I, X₆ is M. In some embodiments of Formula I, X₆ is L. In some embodiments of Formula I, X₆ is I. In some embodiments of Formula I, X₆ is F. In some embodiments of Formula I, X₇ is Q. In some embodiments of Formula I, X₇ is M. In some embodiments of Formula I, X₈ is A. In some embodiments of Formula I, X₈ is N. In some embodiments of Formula I, X₁₀ is selected from K, W, and C. In some embodiments of Formula I, X₁₀ is K. In some embodiments of Formula I, X₁₀ is W. In some embodiments of Formula I, X₁₀ is F. In some embodiments of Formula I, X₁₀ is C. In some embodiments of Formula I, X₁₂ is L or E. In some embodiments of Formula I, X₁₂ is L. In some embodiments of Formula I, X₁₂ is T. In some embodiments of Formula I, X₁₂ is Q. In some embodiments of Formula I, X₁₂ is E. In some embodiments of Formula I, X₁₂ is A.

In some embodiments, the AIP comprises an amino acid sequence according to any one of SEQ ID NOs 3 to 26 (see Tables 1 and 4). In some embodiments, the AlP comprises an amino acid sequence according to SEQ ID NO: 3. In some embodiments, the AIP comprises an amino acid sequence according to SEQ ID NO: 4. In some embodiments, the AIP comprises an amino acid sequence according to SEQ ID NO: 5. In some embodiments, the AIP comprises an amino acid sequence according to SEQ ID NO: 6. In some embodiments, the AIP comprises an amino acid sequence according to SEQ ID NO: 7. In some embodiments, the AIP comprises an amino acid sequence according to SEQ ID NO: 8. In some embodiments, the ATP comprises an amino acid sequence according to SEQ ID NO: 9. In some embodiments, the AIP comprises an amino acid sequence according to SEQ ID NO: 10. In some embodiments, the AIP comprises an amino acid sequence according to SEQ ID NO: 11. In some embodiments, the AlP comprises an amino acid sequence according to SEQ ID NO: 12. In some embodiments, the AIP comprises an amino acid sequence according to SEQ ID NO: 13. In some embodiments, the AIP comprises an amino acid sequence according to SEQ ID NO: 14. In some embodiments, the AIP comprises an amino acid sequence according to SEQ ID NO: 15. In some embodiments, the AIP comprises an amino acid sequence according to SEQ ID NO: 16. In some embodiments, the AIP comprises an amino acid sequence according to SEQ ID NO: 17. In some embodiments, the AIP comprises an amino acid sequence according to SEQ ID NO: 18. In some embodiments, the AIP comprises an amino acid sequence according to SEQ ID NO: 19. In some embodiments, the AIP comprises an amino acid sequence according to SEQ ID NO: 20. In some embodiments, the AIP comprises an amino acid sequence according to SEQ ID NO: 21. In some embodiments, the AIP comprises an amino acid sequence according to SEQ ID NO: 22. In some embodiments, the AIP comprises an amino acid sequence according to SEQ ID NO: 23. In some embodiments, the AIP comprises an amino acid sequence according to SEQ ID NO: 24. In some embodiments, the AIP comprises an amino acid sequence according to SEQ ID NO: 25. In some embodiments, the AIP comprises an amino acid sequence according to SEQ ID NO: 26.

In some embodiments, the AIP elicits release of at least one anti-inflammatory cytokine from an immune cell. In some embodiments, the at least one anti-inflammatory cytokine is selected from interleukin 10 (IL-10), interleukin 4 (IL-4), interleukin 13 (IL-13), interleukin 22 (IL-22), interleukin 6 (IL-6), and transforming growth factor beta (TGF-β). In some embodiments, the at least one anti-inflammatory cytokine is IL-10. In some embodiments, the at least one anti-inflammatory cytokine is IL-4. In some embodiments, the at least one anti-inflammatory cytokine is IL-13. In some embodiments, the at least one anti-inflammatory cytokine is IL-22. In some embodiments, the at least one anti-inflammatory cytokine is IL-6. In some embodiments, the at least one anti-inflammatory cytokine is TGF-3. In some embodiments, the AIP elicits release of at least one pro-inflammatory cytokine from an immune cell. In some embodiments, the at least one pro-inflammatory cytokine includes interferon gamma (IFN-γ). In some embodiments, the immune cell is a T cell. In some embodiments, the immune cell is a CD4 positive T cell. In some embodiments, the immune cell is a human CD4 positive T cell.

In some embodiments, the AIP is a therapeutic peptide for treatment of an inflammatory disease. In some embodiments, the AIP is a therapeutic peptide for treatment of an allergy or an allergic disease. In some embodiments, the disease or disorder is associated with one or more anti-inflammatory cytokines. In some embodiments, the disease or disorder is associated with the deficiency of the one or more anti-inflammatory cytokines.

In an aspect, the present disclosure provides an anti-inflammatory peptide (AIP) comprising an amino acid sequence of Formula II:

(Formula II)
L-X₂-X₃-X₄-X₅-X₆-K-L-X₉-X₁₀-X₁₁-X₁₂

wherein X₂ is A, Y, W, V, R, Q, M, L, K, I, H, or F; X₃ is L, W, or F; X₄ is L, W, N, or C; X₅ is A, W, or F; X₆ is P, Y, W, V, T, S, R, Q, N, M, L, I, H, G, F, D, C, or A; X₉ is L or W; X₁₀ is R or S; X₁₁ is R or Q; and X₂ is F, V, T, S, R, Q, P, N, K, I, G, E, D, C, or A.

In some embodiments of Formula II, X₂ is selected from A, Y, W, and F. In some embodiments of Formula II, X₂ is A. In some embodiments of Formula II, X₂ is Y. In some embodiments of Formula II, X₂ is W. In some embodiments of Formula II, X₂ is V. In some embodiments of Formula II, X₂ is R. In some embodiments of Formula II, X₂ is Q. In some embodiments of Formula II, X₂ is M. In some embodiments of Formula II, X₂ is L. In some embodiments of Formula II, X₂ is K. In some embodiments of Formula II, X₂ is I. In some embodiments of Formula II, X₂ is H. In some embodiments of Formula II, X₂ is F. In some embodiments of Formula II, X₃ is L or W. In some embodiments of Formula II, X₃ is L. In some embodiments of Formula II, X₃ is W. In some embodiments of Formula II, X₃ is F. In some embodiments of Formula II, X₄ is L or W. In some embodiments of Formula II, X₄ is L. In some embodiments of Formula II, X₄ is W. In some embodiments of Formula II, X₄ is N. In some embodiments of Formula II, X₄ is C. In some embodiments of Formula II, X₅ is A. In some embodiments of Formula II, X₅ is W. In some embodiments of Formula II, X₅ is F.

In some embodiments of Formula II, X₆ is selected from P, W, M, H, F, and C. In some embodiments of Formula II, X₆ is P. In some embodiments of Formula II, X₆ is Y. In some embodiments of Formula II, X₆ is W. In some embodiments of Formula II, X_{b} is V. In some embodiments of Formula II, X₆ is T. In some embodiments of Formula II, X₆ is S. In some embodiments of Formula II, X₆ is R. In some embodiments of Formula II, X₆ is Q. In some embodiments of Formula II, X₆ is N. In some embodiments of Formula II, X₆ is M. In some embodiments of Formula II, X₆ is L. In some embodiments of Formula II, X₆ is I. In some embodiments of Formula II, X₆ is H. In some embodiments of Formula II, X₆ is G. In some embodiments of Formula II, X₆ is F. In some embodiments of Formula II, X₆ is D. In some embodiments of Formula II, X₆ is C. In some embodiments of Formula II, X₆ is A. In some embodiments of Formula II, X₉ is L. In some embodiments of Formula II, X₉ is W. In some embodiments of Formula II, X₁₀ is R. In some embodiments of Formula II, X₁₀ is S. In some embodiments of Formula II, X₁₁ is R. In some embodiments of Formula II, X₁₁ is Q. In some embodiments of Formula II, X₁₂ is selected from F, P, K, G, E, D, C, and A. In some embodiments of Formula II, X₁₂ is selected from F, K, E, D, and A. In some embodiments of Formula II, X₁₂ is F. In some embodiments of Formula II, F₁₂ is V. In some embodiments of Formula II, F₁₂ is T. In some embodiments of Formula II, F₁₂ is S. In some embodiments of Formula II, F₁₂ is R. In some embodiments of Formula II, F₁₂ is Q. In some embodiments of Formula II, F₁₂ is P. In some embodiments of Formula II, F₁₂ is N. In some embodiments of Formula II, F₁₂ is K. In some embodiments of Formula II, F₁₂ is I. In some embodiments of Formula II, F₁₂ is G. In some embodiments of Formula II, F₁₂ is E. In some embodiments of Formula II, F₁₂ is D. In some embodiments of Formula II, F₁₂ is C. In some embodiments of Formula II, F₁₂ is A.

In some embodiments, the AIP comprises an amino acid sequence according to any one of SEQ ID NOs 2 and 27 to 45 (see Tables 1 and 5). In some embodiments, the AIP comprises an amino acid sequence according to SEQ ID NO: 2. In some embodiments, the ATP comprises an amino acid sequence according to SEQ ID NO: 27. In some embodiments, the AIP comprises an amino acid sequence according to SEQ ID NO: 28. In some embodiments, the AIP comprises an amino acid sequence according to SEQ ID NO: 29. In some embodiments, the AIP comprises an amino acid sequence according to SEQ ID NO: 30. In some embodiments, the AIP comprises an amino acid sequence according to SEQ ID NO: 31. In some embodiments, the AIP comprises an amino acid sequence according to SEQ ID NO: 32. In some embodiments, the AIP comprises an amino acid sequence according to SEQ ID NO: 33. In some embodiments, the AIP comprises an amino acid sequence according to SEQ ID NO: 34. In some embodiments, the AIP comprises an amino acid sequence according to SEQ ID NO: 35. In some embodiments, the AIP comprises an amino acid sequence according to SEQ ID NO: 36. In some embodiments, the AIP comprises an amino acid sequence according to SEQ ID NO: 37. In some embodiments, the AIP comprises an amino acid sequence according to SEQ ID NO: 38. In some embodiments, the AIP comprises an amino acid sequence according to SEQ ID NO: 39. In some embodiments, the AIP comprises an amino acid sequence according to SEQ ID NO: 40. In some embodiments, the AIP comprises an amino acid sequence according to SEQ ID NO: 41. In some embodiments, the AIP comprises an amino acid sequence according to SEQ ID NO: 42. In some embodiments, the AIP comprises an amino acid sequence according to SEQ ID NO: 43. In some embodiments, the AIP comprises an amino acid sequence according to SEQ ID NO: 44. In some embodiments, the AIP comprises an amino acid sequence according to SEQ ID NO: 45.

In some embodiments, the AIP elicits release of at least one anti-inflammatory cytokine from an immune cell. In some embodiments, the at least one anti-inflammatory cytokine is selected from interleukin 10 (IL-10), interleukin 4 (IL-4), interleukin 13 (IL-13), interleukin 22 (IL-22), interleukin 6 (IL-6), and transforming growth factor beta (TGF-β). In some embodiments, the at least one anti-inflammatory cytokine is IL-10. In some embodiments, the at least one anti-inflammatory cytokine is IL-4. In some embodiments, the at least one anti-inflammatory cytokine is IL-13. In some embodiments, the at least one anti-inflammatory cytokine is IL-22. In some embodiments, the at least one anti-inflammatory cytokine is IL-6. In some embodiments, the at least one anti-inflammatory cytokine is TGF-β. In some embodiments, the AIP elicits release of two or more anti-inflammatory cytokines from an immune cell. In some embodiments, the two or more anti-inflammatory cytokines are selected from IL-10, IL-4, IL-13, IL-22, IL-6, and TGF-β. In some embodiments, the AlP elicits release of at least one pro-inflammatory cytokine from an immune cell. In some embodiments, the at least one pro-inflammatory cytokine includes interferon gamma (IFN-γ). In some embodiments, the immune cell is a T cell. In some embodiments, the immune cell is a CD4 positive T cell. In some embodiments, the immune cell is a human CD4 positive T cell.

In some embodiments, the AIP is a therapeutic peptide for treatment of an inflammatory disease. In some embodiments, the AIP is a therapeutic peptide for treatment of an allergy or an allergic disease.

In some embodiments, the present disclosure provides a method of eliciting release of one or more anti-inflammatory cytokines from an immune cell comprising contacting the immune cell with a peptide or an AIP of any one of the preceding embodiments. In some embodiments, the one or more anti-inflammatory cytokines are selected from interleukin 10 (IL-10), interleukin 4 (IL-4), interleukin 13 (IL-13), interleukin 22 (IL-22), interleukin 6 (IL-6), and transforming growth factor beta (TGF-β). In some embodiments, the one or more anti-inflammatory cytokines includes IL-10. In some embodiments, the one or more anti-inflammatory cytokines includes IL-4. In some embodiments, the one or more anti-inflammatory cytokines includes IL-13. In some embodiments, the one or more anti-inflammatory cytokines includes IL-22. In some embodiments, the one or more anti-inflammatory cytokines includes IL-6. In some embodiments, the one or more anti-inflammatory cytokines includes TGF-β. In some embodiments, the immune cell is a T cell. In some embodiments, the immune cell is a CD4 positive T cell. In some embodiments, the immune cell is a human CD4 positive T cell.

In some embodiments, the present disclosure provides a method of using a peptide or an AlP of any one of the preceding embodiments as a positive control for eliciting release of at least one anti-inflammatory cytokine from a cell in an immune stimulating assay. In some embodiments, the anti-inflammatory cytokine is selected from interleukin 10 (IL-10), interleukin 4 (IL-4), interleukin 13 (IL-13), interleukin 22 (IL-22), interleukin 6 (IL-6), transforming growth factor beta (TGF-β), and combinations thereof. In some embodiments, the cell is an immune cell. In some embodiments, the cell is a T cell. In some embodiments, the method comprises contacting the peptide or the AIP with the cell (e.g., immune cell). The contact can be made in vitro or in vivo. In some embodiments, the method comprises detecting the release of the at least one anti-inflammatory cytokine. In some embodiments, the method comprises detecting the presence or concentration of the at least one anti-inflammatory cytokine.

### Methods for Detecting Cytokine Release Induced by Peptides

In an aspect, the present disclosure provides methods for characterizing a cytokine profile induced by a peptide comprising: (a) isolating naïve CD4 positive T cells from peripheral blood mononuclear cells (PBMCs); (b) expanding the isolated naïve CD4 positive T cells; (c) co-culturing the expanded naïve CD4 positive T cells with PBMCs and stimulating the expanded naïve CD4 positive T cells one or more times with the peptide; and (d) detecting a signal corresponding to a level of one or more cytokines produced by the expanded naïve CD4 positive T cells. In an aspect, the present disclosure provides methods for characterizing a cytokine profile induced by a peptide comprising one or more steps selected from (a) isolating naïve CD4 positive T cells from peripheral blood mononuclear cells (PBMCs); (b) expanding the isolated naïve CD4 positive T cells; (c) co-culturing the expanded naïve CD4 positive T cells with PBMCs and stimulating the expanded naïve CD4 positive T cells one or more times with the peptide; and (d) detecting a signal corresponding to a level of one or more cytokines produced by the expanded naïve CD4 positive T cells.

In some embodiments, expanding the isolated naïve CD4 positive T cells leads to at least a 5-fold expansion of the isolated naïve CD4 positive T cells. In some embodiments, expanding the isolated naïve CD4 positive T cells leads to at least a 10-fold expansion of the isolated naïve CD4 positive T cells. In some embodiments, expanding the isolated naïve CD4 positive T cells leads to at least a 20-fold expansion of the isolated naïve CD4 positive T cells. In some embodiments, expanding the isolated naïve CD4 positive T cells leads to at least a 30-fold expansion of the isolated naïve CD4 positive T cells. In some embodiments, expanding the isolated naïve CD4 positive T cells leads to at least a 50-fold expansion of the isolated naïve CD4 positive T cells. In some embodiments, expanding the isolated naïve CD4 positive T cells leads to at least a 100-fold expansion of the isolated naïve CD4 positive T cells.

In some embodiments, the stimulating is performed in the presence of at least one helper cytokine. In some embodiments, the at least one helper cytokine is selected from interleukin-2 (IL-2) and interleukin-7 (IL-7). In some embodiments, the at least one helper cytokine is IL-2. In some embodiments, the at least one helper cytokine is IL-7. In some embodiments, the at least one helper cytokine is present at a concentration of in a range of about 5 units/milliliter (U/mL) to about 50 U/mL. In some embodiments, the at least one helper cytokine is present at a concentration in a range of about 5 U/mL to about 30 U/mL. In some embodiments, the at least one helper cytokine is present at a concentration of about 10 U/mL.

In some embodiments, the stimulating is performed one time. In some embodiments, the stimulating is performed at least two times. In some embodiments, the stimulating is performed at least three times. In some embodiments, the stimulating is performed at least four times. In some embodiments, the stimulating is performed two times. In some embodiments, the stimulating is performed three times. In some embodiments, at least one of the stimulating steps is performed for a time period of about 30 minutes to about 2 weeks. In some embodiments, each of the stimulating steps is performed for a time period of about 2 hours to about 1 week. In some embodiments, each of the stimulating steps is performed for a time period of about 1 day to about 5 days. In some embodiments, at least one of the stimulating steps is performed for a time period of about 1 day to about 3 days. In some embodiments, each of the stimulating steps is performed for a time period of about 1 day to about 3 days. In some embodiments, each of the stimulating steps is performed for a time period of about 1 day. In some embodiments, each of the stimulating steps is performed for a time period of about 3 days. In some embodiments, each of the stimulating steps comprises suspending the expanded naïve CD4 positive T cells in a fresh volume of media and stimulating the expanded naïve CD4 positive T cells with a fresh sample of the peptide. In some embodiments, at least one of the stimulating steps is performed in the presence of about 1 microgram (μg)/mL to about 100 μg/mL of the peptide. In some embodiments, each of the stimulating steps is performed in the presence of about 1 microgram (μg)/mL to about 100 μg/mL of the peptide. In some embodiments, each of the stimulating steps is performed in the presence of about 10 μg/mL to about 50 μg/ml of the peptide. In some embodiments, each of the stimulating steps is performed in the presence of at least about 5 μg/ml of the peptide. In some embodiments, each of the stimulating steps is performed in the presence of at least about 10 μg/ml of the peptide. In some embodiments, each of the stimulating steps is performed in the presence of at least about 40 μg/ml of the peptide. In some embodiments, each of the stimulating steps is performed in the presence of at least about 50 μg/ml of the peptide. In some embodiments, each of the stimulating steps is performed in the presence of about 40 μg/mL of the peptide.

In some embodiments, the co-culturing is performed with an excess of the expanded naïve CD4 positive T cells relative to the PBMCs. In some embodiments, a ratio of the expanded naïve CD4 positive T cells to the PBMCs during the co-culturing ranges from about 2:1 to about 50:1. In some embodiments, a ratio of the expanded naïve CD4 positive T cells to the PBMCs during the co-culturing is at least about 5:1. In some embodiments, a ratio of the expanded naïve CD4 positive T cells to the PBMCs during the co-culturing is at least about 10:1. In some embodiments, a ratio of the expanded naïve CD4 positive T cells to the PBMCs during the co-culturing is at least about 20:1. In some embodiments, a ratio of the expanded naïve CD4 positive T cells to the PBMCs during the co-culturing is at least about 50:1. In some embodiments, a ratio of the expanded naïve CD4 positive T cells to the PBMCs during the co-culturing is about 4:1.

In some embodiments, expanding the isolated naïve CD4 positive T cells comprises exposing the isolated naïve CD4 positive T cells to magnetic beads coated with anti-CD3 antibodies and anti-CD28 antibodies.

In some embodiments, detecting the signal corresponding to the level of the one or more cytokines comprises detecting the signal by an enzyme-linked immunoassay (ELISA). In some embodiments, detecting the signal corresponding to the level of the one or more cytokines comprises detecting the signal by an enzyme-linked immunoassay (ELISpot). In some embodiments, detecting the signal corresponding to the level of the one or more cytokines comprises detecting the signal by a flow cytometry-based assay. In some embodiments, the one or more cytokines detected in the detecting step include one or more anti-inflammatory cytokines. In some embodiments, the one or more anti-inflammatory cytokines are selected from interleukin 10 (IL-10), interleukin 4 (IL-4), interleukin 13 (IL-13), interleukin 22 (IL-22), interleukin 6 (IL-6), and transforming growth factor beta (TGF-β). In some embodiments, the one or more anti-inflammatory cytokines include IL-10. In some embodiments, the one or more anti-inflammatory cytokines include IL-4. In some embodiments, the one or more anti-inflammatory cytokines include IL-13. In some embodiments, the one or more anti-inflammatory cytokines include IL-22. In some embodiments, the one or more anti-inflammatory cytokines include IL-6. In some embodiments, the one or more anti-inflammatory cytokines include TGF-β. In some embodiments, the one or more cytokines detected in the detecting step include one or more pro-inflammatory cytokines. In some embodiments, the one or more pro-inflammatory cytokines include interferon gamma (IFN-γ). In some embodiments, the signal corresponding to a level of one or more cytokines is detected in a media or a cell.

### Methods of Treatment

In an aspect, the present disclosure provides methods of treating or preventing a disease or disorder in a subject in need thereof. In some embodiments, a method of treating or preventing a disease or disorder in a subject in need thereof comprises administering a peptide or an AIP of any one of the preceding embodiments to the subject.

In some embodiments, the disease or disorder is an inflammatory disease. In some embodiments, the disease or disorder is an allergy or allergic disease.

In some embodiments, administering the peptide or the AIP comprises administering a therapeutically effective amount of the peptide or the AlP to the subject. In some embodiments, administering the peptide or the AIP comprises administering the peptide or the AlP to the subject in combination with one or more additional therapeutic agents.

In some embodiments, administering the peptide or the AIP to the subject comprises administering the peptide or the AIP orally. In some embodiments, administering the peptide or the AIP to the subject comprises administering the peptide or the ATP parenterally. In some embodiments, administering the peptide or the AIP to the subject comprises administering the peptide or the AIP subcutaneously. In some embodiments, administering the peptide or the AIP to the subject comprises administering the peptide or the AIP intramuscularly. In some embodiments, administering the peptide or the AIP to the subject comprises administering the peptide or the AIP intravenously. In some embodiments, administering the peptide or the AIP to the subject comprises administering the peptide or the AIP topically. In some embodiments, administering the peptide or the AIP to the subject comprises administering the peptide or the AIP by infusion.

### Pharmaceutical Compositions

In an aspect, the present disclosure provides pharmaceutical compositions for treating or preventing a disease or disorder. In some embodiments, a pharmaceutical composition of the present disclosure comprises a peptide or an AIP of any one of the preceding embodiments. In some embodiments, the pharmaceutical composition further comprises one or more pharmaceutically acceptable carriers and/or excipients.

In some embodiments, the pharmaceutical composition is formulated for oral administration. In some embodiments, the pharmaceutical composition is formulated for parenteral administration. In some embodiments, the pharmaceutical composition is formulated for subcutaneous administration. In some embodiments, the pharmaceutical composition is formulated for intramuscular administration. In some embodiments, the pharmaceutical composition is formulated for intravenous administration. In some embodiments, the pharmaceutical composition is formulated for topical administration. In some embodiments, the pharmaceutical composition is formulated for administration by infusion.

In some embodiments, the disease or disorder is an inflammatory disease. In some embodiments, the disease or disorder is an allergy or an allergic disease. In some embodiments, the disease or disorder is associated with one or more anti-inflammatory cytokines. In some embodiments, the disease or disorder is associated with the deficiency of one or more anti-inflammatory cytokines.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents.

### Examples

### Example 1: Machine Learning Models for Anti-Inflammatory Peptides

Machine learning models for predicting anti-inflammatory peptides were built as described below and in U.S. patent application No. 63/354,006 which is incorporated herein by reference.

### Acquisition of Dataset for Training and Evaluating Models

Datasets to train and evaluate methods and systems as described herein were obtained from the Immune Epitope Database (IEDB). The database contains results from nearly 2 million experiments from over 20,000 publications which span a wide range of immune epitope data including T cell activation and MHC binding data across a wide range of diseases such as infectious, allergic, autoimmune and cancer. The datasets that capture cytokine release from T cells activated with peptide epitopes were the basis for training algorithms as described herein. The anti-inflammatory cytokines selected to train the algorithms included: IL-10, IL-4, IL-13, IL-22, IL-6 and TGF-β. To build preliminary selectivity models, the pro-inflammatory cytokine IFN-γ, which can suppress the anti-inflammatory response, was selected. Moreover, MHC-II binding data was utilized as well to train models to predict whether a peptide would bind to a specific MHC-II allele of a donor derived PBMC sample, a pre-requisite for T cell activation. Common MHC-II alleles that had substantial size datasets to result in sufficient performance were selected to build the allele specific models and are shown in Table 2 below.

The cytokine release data from IEDB was limited to linear peptides, the assays were limited to human cells, and no diseases were excluded. The cytokine release assays in IEDB have qualitative labels (positive/negative) with some studies including the frequency of responding donors. The data is entered via human curation of publications except for high throughput studies that are uploaded programmatically. The sizes of the available datasets are shown in Table 2 below. During model training (e.g., as performed in other Examples), negatives were undersampled to match the number of positives and simplify the interpretation of the performance metrics.

TABLE 2
Datasets for training and evaluating trained algorithms

| | | | Posi- | Nega- |
|---|---|---|---|---|
| Dataset | Cytokines/MHC alleles | Size | tives | tives |
| Anti-inflammatory | IL-10, IL-4, IL-13, | 3021 | 50% | 50% |
| cytokine assays | IL-22, IL-6, TGF-β | | | |
| (AIPs) | | | | |
| Pro-inflammatory | IFN-γ (limited to | 14860 | 50% | 50% |
| cytokine assays | MHC-II dependent) | | | |
| (PIPs) | | | | |
| MHC-II binding | HLA-DRB1: *0101, | 3000- | 13- | 43- |
| | *0301, *0405, *0701, | 17500 | 57% | 87% |
| | *0802, *0901, *1101, | | | |
| | *1501, HLA- | | | |
| | DQA1*0501- | | | |
| | DQB1*0301 | | | |

### Convolutional Neural Network (CNN) Model

A convolutional neural network was established in the tensorflow/keras framework. Peptides were represented as a one hot encoded matrix with 20 channels representing the amino acids. The peptides were zero padded to 24 amino acids as the range of lengths of the peptides in dataset was from 9-24 amino acids. In some cases, additional peptide encodings were utilized corresponding to biophysical amino acid properties. In some cases, peptide encodings were utilized corresponding to the evolutionary amino acid exchange properties.

The CNNs relied on convolutional kernels that scan across the peptide. The kernel weights are learned during model training. In some cases, multiple kernels were utilized to learn several patterns. The kernels scan across the peptide and result in an activation map (initial feature detection). To enable detection of more complex patterns, additional convolutional layers can be implemented where a new set of kernels scan the activation map and learn more complex features. The convolutional layers are then flattened into a vector of nodes and followed by deep/fully connected layers where the resulting nodes are allowed to interact. Finally, the deep layers are followed by an output layer, in this case a single node that outputs a probability between 0-1 that a peptide is an anti-inflammatory peptide (AIP). A threshold of 0.5 was applied to the probability resulting in classification of a peptide as either AIP on not AIP, though other thresholds and multi-class classifications are possible. The model training process comprised passing batches of peptides and their corresponding class labels through the network, comparing the actual labels to the predicted labels to calculate loss/error, backpropagating the gradient and updating the convolutional kernel and deep layer weights. The process is continued until a set number of iterations/epochs is reached, or in the case of early stopping, the validation loss reached a minimum.

### Performance of Trained Algorithms

The model architectures and hyperparameters were optimized using Monte Carlo cross validation with 30 folds. Architecture optimization included optimizing number of layers, number of nodes, kernels, encodings, and activation. Hyperparameter optimization included optimizing learning rate and batch size. The following performance metrics were obtained for the optimized model trained on AIP cytokine release data: area under the receiver operating characteristic curve (AUC) of 0.79 and precision of 0.83. Precision is defined as number of true positives (TP) divided by number of true positives plus number of false positives (FP) (TP/(TP+FP)), and AUC is defined as area under true positive rate vs. false positive rate curve.

### Example 2: Prediction of Peptides with Anti-Inflammatory Activity

Gut metaproteome peptides were downloaded from the Mechanism of Action of the Human Microbiome (MAHMI) database, subset to peptides in the range of 10-20 amino acids and processed by the models as described in Example 1 to predict AIP status and IFN-γ capability. Compared to the documented peptides that only had 0.6 predicted probability of AIP, peptides that had >0.9 probability were identified. Moreover, peptides that showed selectivity towards anti-inflammatory activity (high AIP probability, low IFN-γ probability) were observed. The MAHMI peptides were also processed by an MHC-II binding prediction model as described in Example 1. To facilitate sourcing of PBMCs from multiple donors, the peptides that could bind to the DRB1-1101 allele, a common allele in the population, were prioritized for testing. The predicted probabilities of 10 peptides are shown in Table 3 below. AIP prob is the probability that a peptide that would result in release of anti-inflammatory cytokines. IFN gamma prob is the probability that a peptide would result in IFN gamma release (a pro-inflammatory cytokine). The remaining columns indicate the predicted binding to MHC-II alleles. Peptide KBP58 is predicted as not binding to HLA-DRB1*1101 allele or any other alleles that were present in the donors tested and is expected to be negative for cytokine activation. Peptides KBP63-KBP79 show binding to HLA-DRB1*1 101 allele present in donors tested and show high AIP and IFN gamma probabilities. They are expected to result in release of both anti-inflammatory and pro-inflammatory cytokines with preference for anti-inflammatory cytokines based on the high AIP probabilities. Peptide KBP82 has high specificity towards AIP compared to IFN gamma and is expected to predominantly result in anti-inflammatory cytokine release.

TABLE 3

| | | IFN | | | | | | | | |
|---|---|---|---|---|---|---|---|---|---|---|
| Peptide | AIP | gamma | HLA- | HLA- | HLA- | HLA- | HLA- | HLA- | HLA- | HLA- |
| ID | prob | prob | DRB1*0101 | DRB1*0301 | DRB1*0405 | DRB1*0701 | DRB1*0802 | DRB1*0901 | DRB1*1101 | DRB1*1501 |
| KBP58 | 0.82 | 0.37 | 0 | 0 | 0 | 0 | 0 | 0 | 0 | 0 |
| KBP63 | 0.96 | 0.80 | 1 | 0 | 1 | 1 | 1 | 1 | 1 | 0 |
| KBP64 | 0.95 | 0.83 | 1 | 0 | 1 | 1 | 1 | 0 | 1 | 1 |
| KBP66 | 0.95 | 0.77 | 1 | 0 | 1 | 1 | 1 | 1 | 1 | 1 |
| KBP67 | 0.95 | 0.74 | 1 | 0 | 0 | 0 | 0 | 0 | 1 | 0 |
| KBP70 | 0.94 | 0.79 | 0 | 0 | 0 | 0 | 0 | 0 | 1 | 1 |
| KBP73 | 0.94 | 0.69 | 1 | 1 | 0 | 1 | 1 | 0 | 1 | 1 |
| KBP78 | 0.93 | 0.80 | 1 | 1 | 0 | 1 | 1 | 0 | 1 | 1 |
| KBP79 | 0.93 | 0.92 | 0 | 1 | 0 | 1 | 1 | 0 | 1 | 1 |
| KBP82 | 0.84 | 0.38 | 1 | 0 | 0 | 1 | 0 | 1 | 1 | 0 |

### Example 3: PBMC Assay to Test Activation of T Cells

The traditional assay to test the activation of T cells with peptide antigen consists of stimulating peripheral blood mononuclear cells (PBMCs) 3-4 times with a peptide antigen and measuring cytokine release using ELISA or other assays. PBMCs contain several cell types including cells that can act as antigen presenting cells (APC) that bind and present the peptide on the MHC complex and T cells that interact with the peptide-MHC complex. The first step of the biological cascade is governed by the specificity of the peptide interaction with the MHC-II complex allele. An individual can express up to 6 MHC-II alleles. The second step of the biological cascade is governed by the specificity of the T cell receptor (TCR) that binds to the peptide-MHC complex. Humans express a large repertoire of >1×10⁷ TCR clones. Upon productive interaction with the peptide-MHC-II complex, the CD4 positive T cells are activated and release cytokines that signal the state of the cells and promote further proliferation and differentiation. The release of various cytokines can be measured in cell supernatants using commercially available ELISA kits.

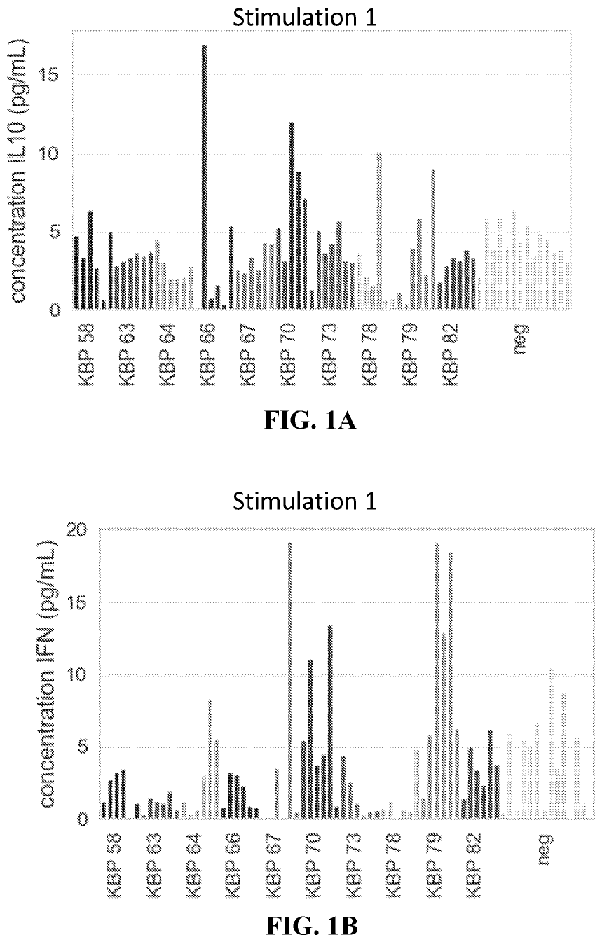

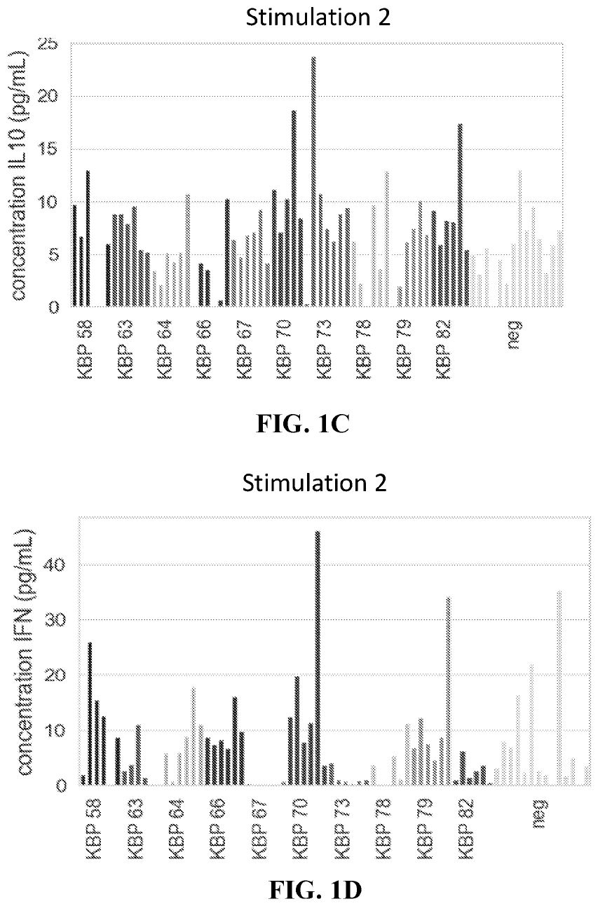

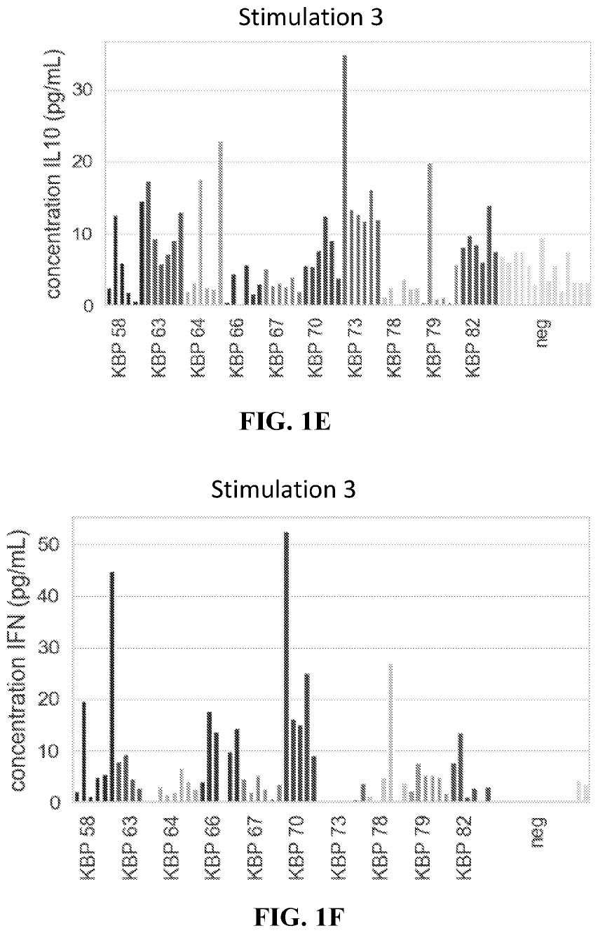

Cryopreserved PBMCs were sourced from StemExpress with HLA profiles containing the HLA-DRB1*1101 that all the peptides except KBP58 are predicted to bind. The thawed PBMCs were rested in serum containing Roswell Park Memorial Institute (RPMI) medium for 24 hours, aliquoted into 24 well plates and stimulated with the peptide at 40 micrograms per milliliter (μg/mL) every 3 days. Low level IL-2 was present to maintain viability and promote proliferation. Supernatant was collected and IL-10 levels quantified using ELISA. The cytokine levels for IL-10 and IFN-γ in individual wells after each of three stimulations are shown in FIGS. 1A-1F.

Large well to well variation was observed. The well-to-well variation stems from the very low frequency of antigen specific naïve T cells in PBMCs which is estimated to be 0.001%-0.01%. Thus in 1 well with 1 million PBMCs, there may only be ˜10 naïve T cells that bear a TCR clone with sufficient antigen specificity to form a potential interaction with a peptide displayed on MHC-II by an APC.

The use of the traditional PBMC assay makes it challenging to identify peptides that are anti-inflammatory or pro-inflammatory due to the high well-to-well variation. The negative controls show some self-activation increasing background signal. The peptide that does not bind donor alleles KBP5S8 shows activation indicating the assay may be unreliable. The cell viability after 3 peptide stimulations was also observed to decrease.

### Example 4: Expanded CD4 Positive (CD4+) T Cell Assay Development

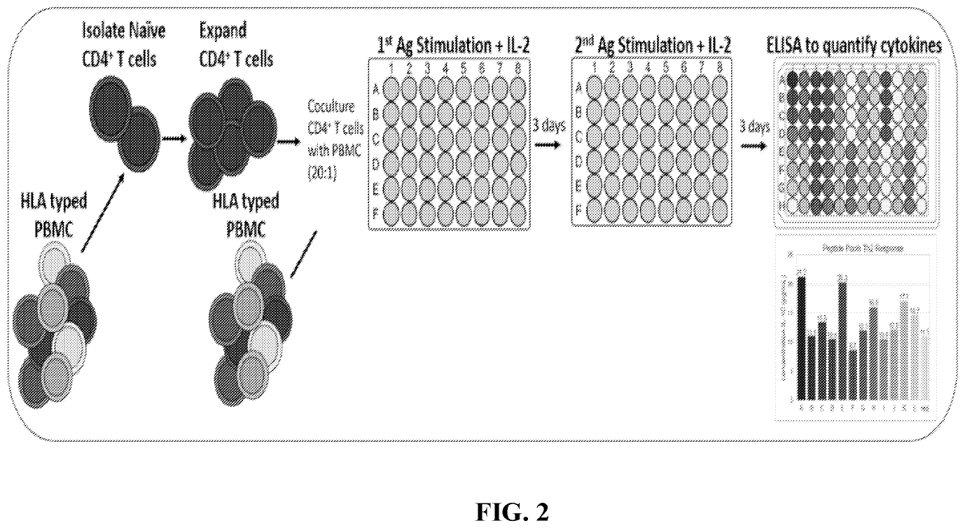

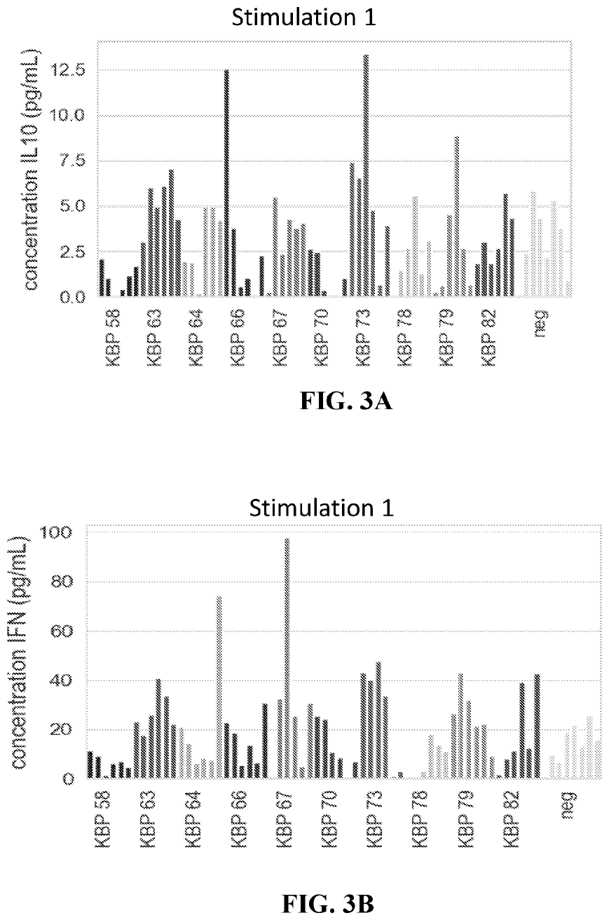

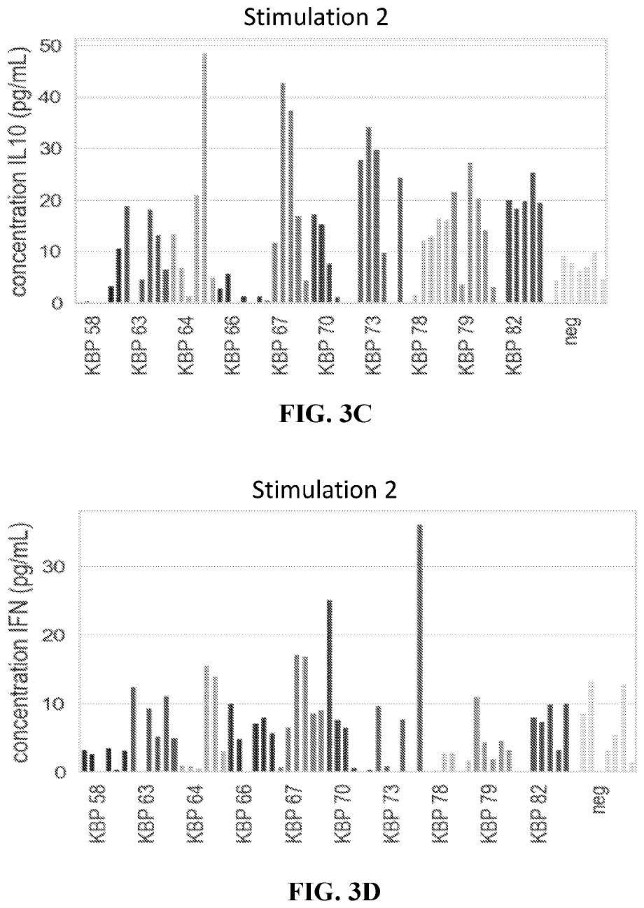

To improve the robustness of the assay and decrease the well-to-well variation, a novel assay system was developed. In effect, by expanding the naïve CD4+ T cells, the number of antigen specific T cells can be increased per well with the goal of detecting a responding well with greater frequency. An expanded CD4+ T cell assay is shown in FIG. 2. To expand the naïve CD4+ T cells, the cells are first isolated from PBMCs using negative selection with antibody coated magnetic beads. The naïve CD4+ T cells are then polyclonally expanded using anti-CD3 and anti-CD28 Dynabeads. An expansion of up to 20-fold was observed. The beads were then removed, and the cells were rested for 24 hours. The expanded CD4+ T cells were then co-cultured with PBMCs at a 4:1 ratio and stimulated with peptides twice every 3 days. IL-2 helper cytokine was included. Peptides were utilized at 40 μg/mL. The cytokine levels (IL-10 and IFN-γ) in individual wells after each stimulation are shown in FIGS. 3A-3D. After two peptide stimulations, IL-10 levels in the range of 20-50 picograms (μg)/mL were observed as compared to 5-10 pg/mL for negative controls (vehicle only) (see FIG. 3C).

To demonstrate that the assay can detect naïve CD4+ T cells responses, the naïve antigen Keyhole limpet haemocyanin KLH, which results in IFN-γ release, was utilized as a control. Strong IFN-γ signal was observed, with 80 μg/mL upon single and 150 μg/mL upon double stimulation.

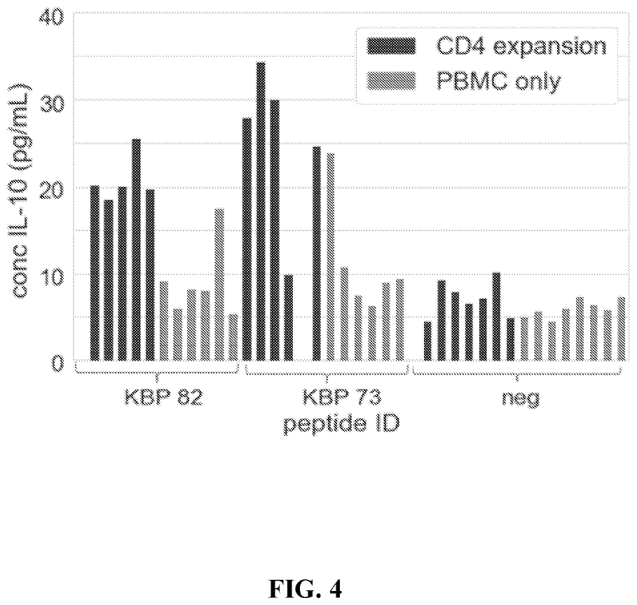

A comparison of IL-10 signal and well-to-well variation upon two peptide stimulations using the expanded CD4+ T cell assay (CD4 expansion) and the traditional assay (PBMC only) is shown in FIG. 4. The expanded CD4+ T cell assay showed improved well-to-well variability, higher signal, and required only two stimulations. Thus, the expanded CD4+ T cell assay overcomes significant challenges and results in robust and reproducible signals.

### Example 5: Expanded CD4+ T Cell Assay Protocol

### PBMC:

PBMCs were isolated from healthy HLA typed donors of different ages, ethnicities, and genders. Donors possessing specific DRB1 alleles, expected to bind to each target peptide antigen, are chosen. 100 million PBMC were thawed into media (Rosewell Park Memorial Institute (RPMI) 1640 medium, 10% fetal bovine serum (FBS) Heat Inactivated, 1% Penicillin-Streptomycin) and rested at 37° C., 5% CO₂ overnight.

### Peptide Synthesis:

Peptides were synthesized by Vivitide as custom peptide arrays. Peptides were solubilized in sterile water with the addition of dimethyl sulfoxide (DMSO) if not completely dissolved in water only.

### T Cell Isolation and Expansion:

Naïve CD4+ T cells are isolated from rested PBMC using the BioLegend MojoSort™ Human CD4 T Cell Isolation Kit. Isolated T cells were counted and cultured at a density of 1×10⁶ cells/mL of media with 30 units per milliliter (U/mL) of IL-2. T cells were activated and expanded using ThermoFisher Dynabeads™ Human T-Activator CD3/CD28. Beads were added at a 1:1 bead-to-cell ratio. T cells are incubated at 37° C., 5% CO₂ for approximately 1 week, splitting in order to maintain the 1×10⁶ cells/mL seeding density and with the addition of fresh media. T cells often saw a 20- to 30-fold expansion.

### Stimulation Assay:

Expansion beads were magnetically removed from the T cell culture. Remaining T cells were cultured at 1×10⁶ cells/mL in media and the IL-2 level was dropped to 10 units/milliliter (U/mL). T cells are rested 24 hours. Meanwhile, another vial of PBMC of the same donor was thawed and similarly rested overnight in media. 1 million expanded T cells and 0.25 million PBMC were cocultured in 1 mL of media containing 10 U/mL IL-2 in 24 well plates. Each well was stimulated with 40 μg of peptide with at least 6 well replicates for each peptide. Sterile water with 10% DMSO was used as a negative control. Plates were incubated at 37° C., 5% CO₂ for 3 days. After 3 days, supernatants of each well were collected into Eppendorf tubes and frozen. Cells were resuspended in fresh media containing 10 U/mL IL-2. Cells were restimulated with 40 μg of peptide in the same format. Plates were incubated for another 3 days (see FIG. 2). Supernatants were collected after the second stimulation and cell culture was terminated.

### Cytokine Quantification:

IL-10 and IFN-γ cytokine concentrations were determined for samples. 96-well microwell plates were coated with capture antibody and incubated overnight at 4° C. Frozen supernatants were thawed and assayed using ELISA MAX™ Deluxe kits.

### Example 6: Quantification of Peptide Response

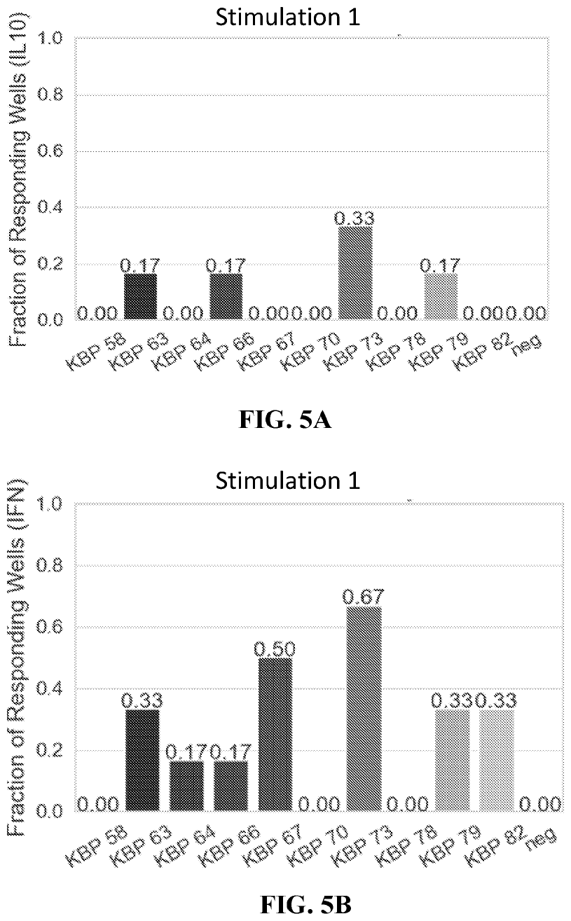

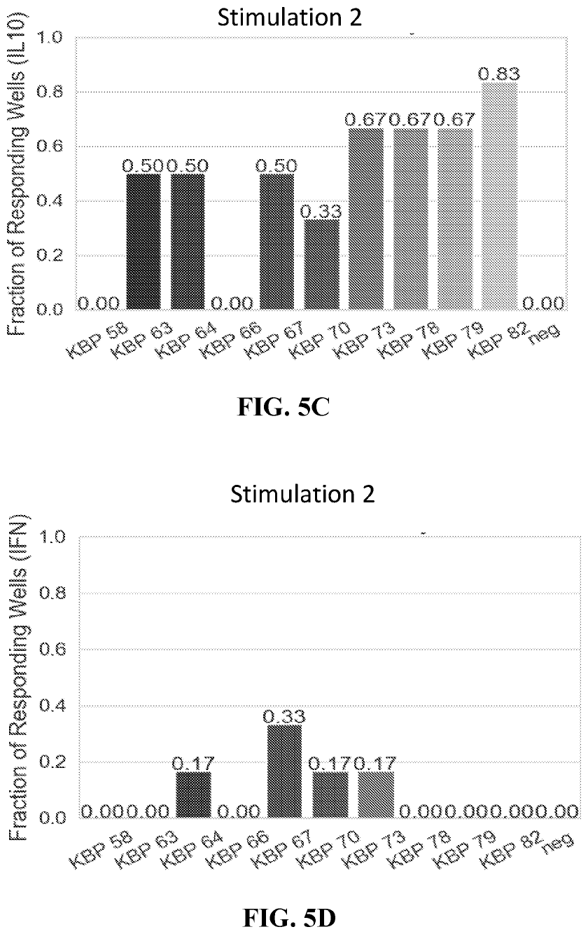

To quantify the number of responding wells for each peptide, a one sample t-test was utilized where signal from each well was compared against the distribution of negative wells from vehicle only controls. The negative distribution was normal (based on Shapiro Wilk test). Wells that were statistically significantly higher in signal than negative distribution after the stringent Bonferroni correction for multiple hypothesis testing were considered as positive wells. As such, for each peptide, a fraction of responding wells was derived. The fraction of responding wells after the first and second peptide stimulations in the expanded CD4+ T cell assay are shown in FIGS. 5A-5D. Moreover, the fraction of responding wells was further subjected to the statistical test for proportions which takes into account the sample size.

Referring to FIGS. 5A-5D, eight peptides have statistically significant fraction of responding wells with 7 peptides (KBP63, KBP64, KBP67, KBP73, KBP78, KBP79, and KBP82) showing >=0.5 fraction responding wells for IL-10 upon second stimulation (see FIG. 5C). IFN-γ release was observed after the first stimulation with some of the peptides however it decreased upon second stimulation which is consistent with the suppressive role of IL-10 as it begins to be produced. Moreover, the KBP82 peptide with high predicted IL-10 specificity compared to IFN-γ showed minimal levels of IFN-γ release.

### Example 7: Comparison of Assay Performance

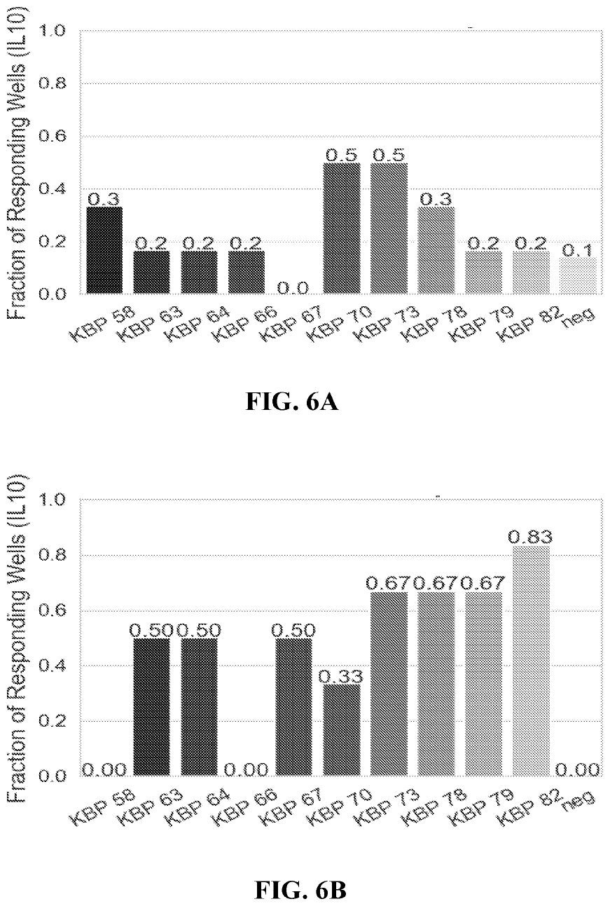

The fraction of responding wells for IL-10 is compared between the PBMC assay and the expanded CD4+ T cell assay in FIGS. 6A-6B. The PBMC assay resulted in only 2 responding peptides after the second stimulation (see FIG. 6A), whereas the expanded CD4+ T cell assay resulted in 8 responding peptides after the second stimulation, with 7 peptides showing strong responses at >=0.5 fraction responding wells (see FIG. 6B).

### Example 8: Testing of Peptides Across Donors

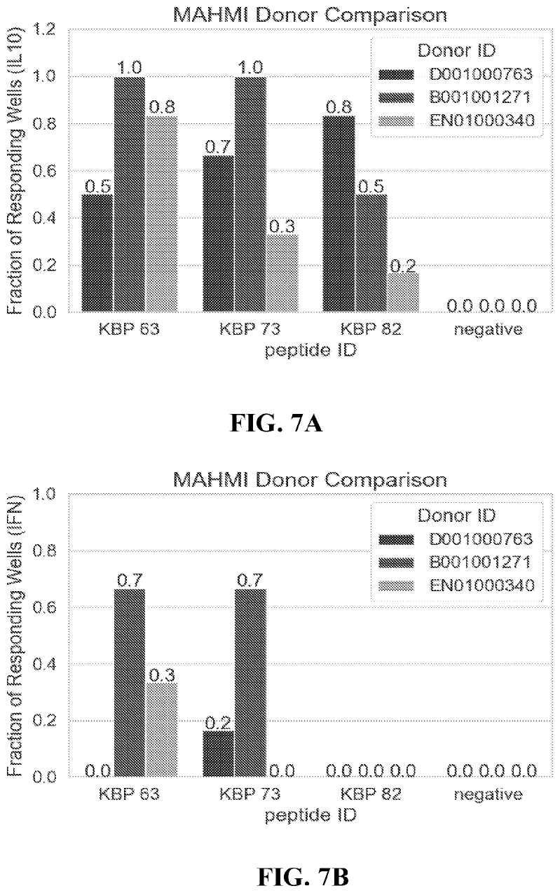

Peptides were tested with three different donors that express the HLA-DRB1*1 101 allele. The frequency of responding wells was derived for each peptide for IL-10 and IFN-γ. As shown in FIGS. 7A-7B, three out of the seven peptides (KBP63 (SEQ ID NO: 1), KBP73 (SEQ ID NO: 2), and KBP82 (SEQ ID NO: 3)) were found to respond across donors. KBP82 showed high specificity for IL-10 production vs IFN-γ as was predicted by the models. The peptides have less than 60% sequence homology to IEBD peptides indicating their novelty. The sequences of the three novel peptides are provided in Table 1.

### Example 9: Prediction of Substitutions to Template AIPs

Having developed a more robust assay to quantify the changes in IL-10 signal and having identified template AIPs (see Example 8 and Table 1), the machine learning models were then utilized to predict substitutions to template peptides KBP82 (SEQ ID NO: 3) and KBP73 (SEQ ID NO: 2).

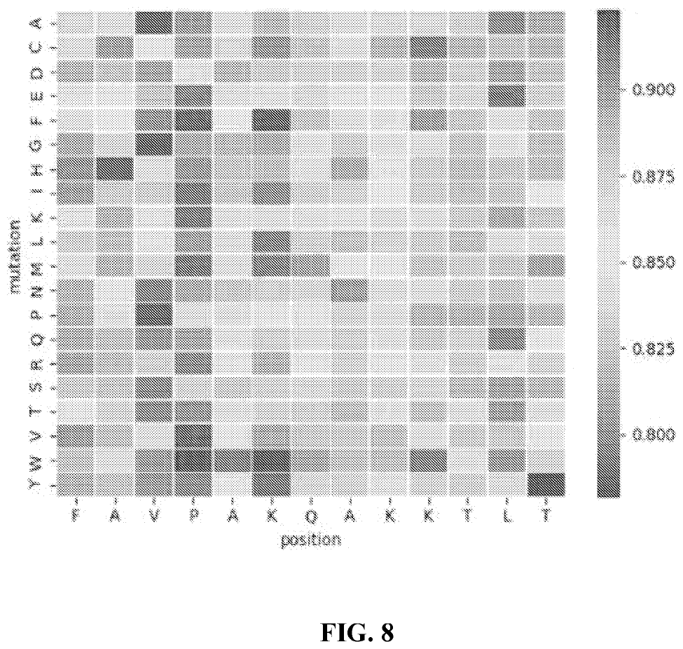

FIG. 8 shows a heatmap of the predicted probabilities for anti-inflammatory activity for all single amino acid substitutions at each position of KBP82, with decreases in probabilities being shown with more blue hue and increases in probabilities being shown with more red hue in the heatmap. The results demonstrate that positions 2 and 3 of KBP82 are sensitive to substitutions, while substitutions at positions 4 and 6 may result in increased in activity. Selected peptide sequences with higher predicted anti-inflammatory activity based on the heatmap are listed in Table 4. The peptide sequences of Table 4 include amino acid substitutions at positions 4 and 6 that have higher predicted anti-inflammatory activity.

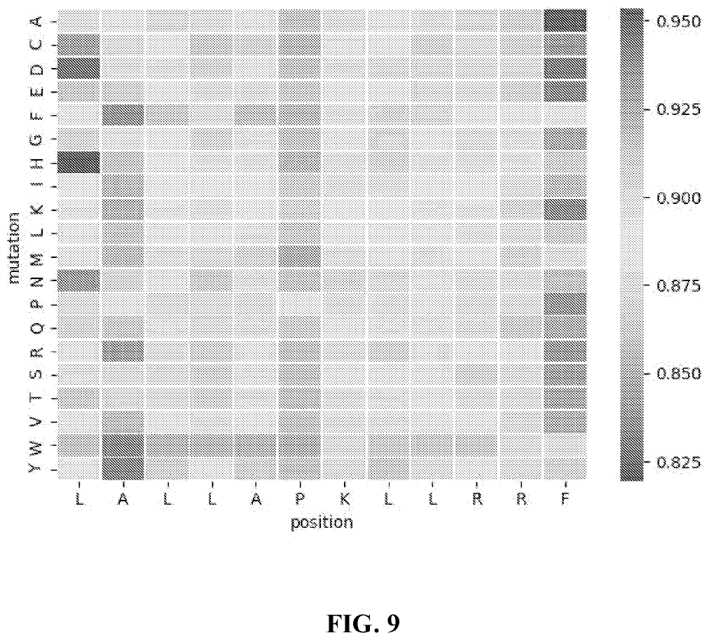

FIG. 9 shows a heatmap of the predicted probabilities of all single amino acid substitutions at each position of KBP73, with decreases in probability for anti-inflammatory activity being shown with more blue hue and increases in probability for anti-inflammatory activity being shown with more red hue. Selected mutated peptide sequences with higher predicted anti-inflammatory activity from the heatmap are listed in Table 5. The peptide sequences of Table 5 include amino acid substitutions at positions 2 and 12 that have higher predicted anti-inflammatory activity.

TABLE 4
Selected Peptide Sequences with Predicted
Anti-Inflammatory Activity Based on
Heatmap of KBP82

| Peptide | Sequence | SEQ ID NO |
|---|---|---|
| KBP82 (P4W) | FAVWAKQAKKTLT | 4 |
| KBP82 (P4V) | FAVVAKQAKKTLT | 5 |
| KBP82 (P4M) | FAVMAKQAKKTLT | 6 |
| KBP82 (P4I) | FAVIAKQAKKTLT | 7 |
| KBP82 (P4F) | FAVFAKQAKKTLT | 8 |
| KBP82 (K6Y) | FAVPAYQAKKTLT | 9 |
| KBP82 (K6W) | FAVPAWQAKKTLT | 10 |
| KBP82 (K6F) | FAVPAFQAKKTLT | 11 |
| KBP82 (P4W, K6Y) | FAVWAYQAKKTLT | 12 |
| KBP82 (P4W, | FAVWAWQAKKTLT | 13 |
| K6W) | | |
| KBP82 (P4W, K6F) | FAVWAFQAKKTLT | 14 |
| KBP82 (P4V, K6Y) | FAVVAYQAKKTLT | 15 |
| KBP82 (P4V, K6W) | FAVVAWQAKKTLT | 16 |
| KBP82 (P4V, K6F) | FAVVAFQAKKTLT | 17 |
| KBP82 (P4M, K6Y) | FAVMAYQAKKTLT | 18 |
| KBP82 (P4M, | FAVMAWQAKKTLT | 19 |
| K6W) | | |
| KBP82 (P4M, K6F) | FAVMAFQAKKTLT | 20 |
| KBP82 (P4I, K6Y) | FAVIAYQAKKTLT | 21 |
| KBP82 (P4I, K6W) | FAVIAWQAKKTLT | 22 |
| KBP82 (P4I, K6F) | FAVIAFQAKKTLT | 23 |
| KBP82 (P4F, K6Y) | FAVFAYQAKKTLT | 24 |
| KBP82 (P4F, K6W) | FAVFAWQAKKTLT | 25 |
| KBP82 (P4F, K6F) | FAVFAFQAKKTLT | 26 |

TABLE 5
Selected Peptide Sequences with Predicted
Anti-Inflammatory Activity Based on
Heatmap of KBP73

| Peptide | Sequence | SEQ ID NO |
|---|---|---|
| KBP73 (A2Y) | LYLLAPKLLRRF | 27 |
| KBP73 (A2W) | LWLLAPKLLRRF | 28 |
| KBP73 (A2F) | LFLLAPKLLRRF | 29 |
| KBP73 (F12K) | LALLAPKLLRRK | 30 |
| KBP73 (F12E) | LALLAPKLLRRE | 31 |
| KBP73 (F12D) | LALLAPKLLRRD | 32 |
| KBP73 (F12A) | LALLAPKLLRRA | 33 |
| KBP73 (A2Y, | LYLLAPKLLRRK | 34 |
| F12K) | | |
| KBP73 (A2Y, | LYLLAPKLLRRE | 35 |
| F12E) | | |
| KBP73 (A2Y, | LYLLAPKLLRRD | 36 |
| F12D) | | |
| KBP73 (A2Y, | LYLLAPKLLRRA | 37 |
| F12A) | | |
| KBP73 (A2W, | LWLLAPKLLRRK | 38 |
| F12K) | | |
| KBP73 (A2W, | LWLLAPKLLRRE | 39 |
| F12E) | | |
| KBP73 (A2W, | LWLLAPKLLRRD | 40 |
| F12D) | | |
| KBP73 (A2W, | LWLLAPKLLRRA | 41 |
| F12A) | | |
| KBP73 (A2F, F12K) | LFLLAPKLLRRK | 42 |
| KBP73 (A2F, F12E) | LFLLAPKLLRRE | 43 |
| KBP73 (A2F, F12D) | LFLLAPKLLRRD | 44 |
| KBP73 (A2F, F12A) | LFLLAPKLLRRA | 45 |

### Example 10: Peptide Synthesis

Synthesis and cleavage: Peptides were synthesized by solid phase peptide synthesis using a Wang resin and Fmoc chemistry with standard protections of amino acids. O-(1H-6-Chlorobenzotriazole-1-yl)-1,1,3,3-tetramethyluronium hexafluorophosphate (HCTU) and N,N-diisopropylethylamine (DIEA) were used to activate the couplings. Dimethylformamide (DMF) was used as the main solvent and for washings. Piperidine (20%) in DMF was used for Fmoc deprotections. Cleavage from the resin was carried out in 90% trifluoroacetic acid (TFA), 2.5% thioanisole, 2.5% ethyl methyl sulfide, and 2.5% ethanedithiol over three hours. Peptides are then dried under nitrogen, solubilized in 50/50 acetonitrile/water, frozen at −80° C., and lyophilized.

Workup: Peptides are solubilized and desalted, and scavengers/protecting groups are removed by a 96 well solid phase extraction plate. Peptides are loaded and washed several times. From there, peptides are eluted from the plate and frozen and lyophilized while data is running. Once data is complete, the peptides are analyzed by quality control.

What is claimed is:

1. A peptide, wherein the peptide comprises an amino acid sequence at least 80% identical to an amino acid sequence selected from KLAKRLLLSMNQ (SEQ ID NO: 1) and FAVPAKQAKKTLT (SEQ ID NO: 3), or an amino acid sequence comprising
(SEQ ID NO: 2)
LALLAPKLLRRF.

2. The peptide of claim 1, wherein the peptide comprises an amino acid sequence selected from the group consisting of KLAKRLLLSMNQ (SEQ ID NO: 1), LALLAPKLLRRF (SEQ ID NO: 2), and FAVPAKQAKKTLT (SEQ ID NO: 3).

3. The peptide of claim 1, wherein the peptide comprises the amino acid sequence
(SEQ ID NO: 1)
KLAKRLLLSMNQ.

4. The peptide of claim 1, wherein the peptide consists of the amino acid sequence
(SEQ ID NO: 1)
KLAKRLLLSMNQ.

5. The peptide of claim 1, wherein the peptide comprises the amino acid sequence
(SEQ ID NO: 2)
LALLAPKLLRRF.

6. The peptide of claim 1, wherein the peptide consists of the amino acid sequence
(SEQ ID NO: 2)
LALLAPKLLRRF.

7. The peptide of claim 1, wherein the peptide comprises the amino acid sequence
(SEQ ID NO: 3)
FAVPAKQAKKTLT.

8. The peptide of claim 1, wherein the peptide consists of the amino acid sequence
(SEQ ID NO: 3)
FAVPAKQAKKTLT.

9. The peptide of any one of claims 1 to 8, wherein the peptide elicits release of at least one anti-inflammatory cytokine from an immune cell.

10. The peptide claim 9, wherein the at least one anti-inflammatory cytokine is selected from interleukin 10 (IL-10), interleukin 4 (IL-4), interleukin 13 (IL-13), interleukin 22 (IL-22), interleukin 6 (IL-6), and transforming growth factor beta (TGF-β).

11. The peptide of claim 9, wherein the at least one anti-inflammatory cytokine is interleukin 10 (IL-10).

12. The peptide of any one of claims 9 to 11, wherein the immune cell is a CD4 positive T cell.

13. The peptide of any one of claims 9 to 12, wherein the immune cell is a human CD4 positive T cell.

14. The peptide of any one of claims 9 to 13, wherein the peptide is a therapeutic peptide for treatment of an inflammatory disease.

15. The peptide of any one of claims 9 to 13, wherein the peptide is a therapeutic peptide for treatment of an allergy or an allergic disease.

16. An anti-inflammatory peptide (AIP) comprising an amino acid sequence of KLAKRLLLSMNQ (SEQ ID NO: 1), LALLAPKLLRRF (SEQ ID NO: 2), FAVPAKQAKKTLT (SEQ ID NO: 3), or an amino acid sequence that has 1, 2, or 3 amino acid substitutions, additions, or deletions relative to the amino acid sequences of KLAKRLLLSMNQ (SEQ ID NO: 1), LALLAPKLLRRF (SEQ ID NO: 2), or FAVPAKQAKKTLT (SEQ ID NO: 3), wherein the AIP consists of 10-16 amino acid residues.

17. The AIP of claim 16, wherein the AIP comprises an amino acid sequence of KLAKRLLLSMNQ (SEQ ID NO: 1), LALLAPKLLRRF (SEQ ID NO: 2), FAVPAKQAKKTLT (SEQ ID NO: 3), or an amino acid sequence that has at most 1 amino acid substitution, addition, or deletion relative to the amino acid sequence of
(SEQ ID NO: 1)
KLAKRLLLSMNQ,
(SEQ ID NO: 2)
LALLAPKLLRRF,
or
(SEQ ID NO: 3)
FAVPAKQAKKTLT.

18. The AIP of claim 16, wherein the AIP comprises an amino acid sequence of
(SEQ ID NO: 1)
KLAKRLLLSMNQ,
(SEQ ID NO: 2)
LALLAPKLLRRF,
or
(SEQ ID NO: 3)
FAVPAKQAKKTLT.

19. The AIP of claim 16, wherein the AIP comprises the amino acid sequence of
(SEQ ID NO: 1)
KLAKRLLLSMNQ.

20. The AIP of claim 16, wherein the AIP consists of the amino acid sequence of
(SEQ ID NO: 1)
KLAKRLLLSMNQ.

21. The AIP of claim 16, wherein the AIP comprises the amino acid sequence of
(SEQ ID NO: 2)
LALLAPKLLRRF.

22. The AIP of claim 16, wherein the AIP consists of the amino acid sequence of
(SEQ ID NO: 2)
LALLAPKLLRRF.

23. The AIP of claim 16, wherein the AIP comprises the amino acid sequence of
(SEQ ID NO: 3)
FAVPAKQAKKTLT.

24. The AIP of claim 16, wherein the AIP consists of the amino acid sequence of
(SEQ ID NO: 3)
FAVPAKQAKKTLT.

25. The AIP of any one of claims 16 to 24, wherein the AIP elicits release of at least one anti-inflammatory cytokine from a CD4 positive T cell.

26. The AIP of claim 25, wherein the at least one anti-inflammatory cytokine is interleukin 10 (IL-10).

27. The AIP of claim 25, wherein the at least one anti-inflammatory cytokine is selected from interleukin 10 (IL-10), interleukin 4 (IL-4), interleukin 13 (IL-13), interleukin 22 (IL-22), interleukin 6 (IL-6), and transforming growth factor beta (TGF-β).

28. The AIP of any one of claims 16 to 27, wherein the AIP is a therapeutic peptide for treatment of an inflammatory disease.

29. The AIP of any one of claims 16 to 27, wherein the AIP is a therapeutic peptide for treatment of an allergy or an allergic disease.

30. An anti-inflammatory peptide (AIP) comprising an amino acid sequence of Formula I,
(Formula I)
X₁-A-X₃-X₄-X₅-X₆-X₇-X₈-K-X₁₀-T-X₁₂-T,
wherein
X₁ is F or H;
X₃ is V, Y, W, or F;
X₄ is P, Y, W, V, T, R, M, K, I, H, F, or E;
X₅ is A or W;
X₆ is K, Y, W, M, L, I, or F;
X₇ is Q or M;
X₈ is A or N;
X₁₀ is K, W, F, or C; and
X₁₂ is L, T, Q, E, or A.

31. The AIP of claim 30, wherein X₁ is F.

32. The AIP of claim 30 or 31, wherein X₃ is V.

33. The AIP of any one of claims 30 to 32, wherein X₄ is selected from P, W, V, M, I, and F.

34. The AIP of claim 33, wherein X₄ is P.

35. The AIP of any one of claims 30 to 34, wherein X₅ is A.

36. The AIP of any one of claims 30 to 35, wherein X₆ is selected from K, Y, W, and F.

37. The AIP of claim 36, wherein X₆ is K.

38. The AIP of any one of claims 30 to 37, wherein X₇ is Q.

39. The AIP of any one of claims 30 to 38, wherein X₈ is A.

40. The AIP of any one of claims 30 to 39, wherein X₁₀ is selected from K, W, and C.

41. The AIP of claim 40, wherein X₁₀ is K.

42. The AIP of any one of claims 30 to 41, wherein X₁₂ is L or E.

43. The AIP of claim 42, wherein X₁₂ is L.

44. The AIP of claim 30, wherein the AIP comprises an amino acid sequence according to any one of SEQ ID NOs 3-26.

45. The AIP of any one of claims 30 to 44, wherein the AIP elicits release of at least one anti-inflammatory cytokine from an immune cell.

46. The AIP of claim 45, wherein the at least one anti-inflammatory cytokine is selected from interleukin 10 (IL-10), interleukin 4 (IL-4), interleukin 13 (IL-13), interleukin 22 (IL-22), interleukin 6 (11L-6), and transforming growth factor beta (TGF-β).

47. The AIP of claim 45, wherein the at least one anti-inflammatory cytokine is interleukin 10 (IL-10).

48. The AIP of any one of claims 45 to 47, wherein the immune cell is a CD4 positive T cell.

49. The AIP of any one of claims 45 to 48, wherein the immune cell is a human CD4 positive T cell.

50. The AIP of any one of claims 45 to 49, wherein the AIP is a therapeutic peptide for treatment of an inflammatory disease.

51. The AIP of any one of claims 45 to 49, wherein the AIP is a therapeutic peptide for treatment of an allergy or an allergic disease.

52. An anti-inflammatory peptide (AIP) comprising an amino acid sequence of Formula II,
(Formula II)
L-X₂-X₃-X₄-X₅-X₆-K-L-X₉-X₁₀-X₁₁-X₁₂
wherein
X₂ is A, Y, W, V, R, Q, M, L, K, I, H, or F;
X₃ is L, W, or F;
X₄ is L, W, N, or C;
X₅ is A, W, or F;
X₆ is P, Y, W, V, T, S, R, Q, N, M, L, I, H, G, F, D, C, or A;
X₉ is L or W;
X₁₀ is R or S;
X₁₁ is R or Q; and
X₁₂ is F, V, T, S, R, Q, P, N, K, I, G, E, D, C, or A.

53. The AIP of claim 52, wherein X₂ is selected from A, Y, W, and F.

54. The AIP of claim 53, wherein X₂ is A.

55. The AIP of any one of claims 52 to 54, wherein X₃ is L or W.

56. The AIP of claim 55, wherein X₃ is L.

57. The AIP of any one of claims 52 to 56, wherein X₄ is L or W.

58. The AIP of claim 57, wherein X₄ is L.

59. The AIP of any one of claims 52 to 58, wherein X₅ is A.

60. The AIP of any one of claims 52 to 59, wherein X₆ is selected from P, W, M, H, F, and C.

61. The AIP of claim 60, wherein X₆ is P.

62. The AIP of any one of claims 52 to 61, wherein X₉ is L.

63. The AIP of any one of claims 52 to 61, wherein X₁₀ is R.

64. The AIP of any one of claims 52 to 63, wherein X₁₁ is R.

65. The AIP of any one of claims 52 to 64, wherein X₁₂ is selected from F, P, K, G, E, D, C, and A.

66. The AIP of claim 65, wherein X₁₂ is selected from F, K, E, D, and A.

67. The AIP of claims 65 or 66, wherein X₁₂ is F.

68. The AIP of claim 52, wherein the AIP comprises an amino acid sequence selected from any one of SEQ ID NOs 2 and 27-45.

69. The AIP of any one of claims 52 to 68, wherein the AIP elicits release of at least one anti-inflammatory cytokine from an immune cell.

70. The AIP of claim 69, wherein the at least one anti-inflammatory cytokine is selected from interleukin 10 (IL-10), interleukin 4 (IL-4), interleukin 13 (IL-13), interleukin 22 (IL-22), interleukin 6 (IL-6), and transforming growth factor beta (TGF-β).

71. The AIP of claim 69, wherein the at least one anti-inflammatory cytokine is interleukin 10 (IL-10).

72. The AIP of any one of claims 69 to 71, wherein the immune cell is a CD4 positive T cell.

73. The AIP of any one of claims 69 to 71, wherein the immune cell is a human CD4 positive T cell.

74. The AIP of any one of claims 69 to 73, wherein the AIP is a therapeutic peptide for treatment of an inflammatory disease.

75. The AIP of any one of claims 69 to 73, wherein the AIP is a therapeutic peptide for treatment of an allergy or an allergic disease.

76. A method for characterizing a cytokine profile induced by a peptide, comprising:
(a) isolating naïve CD4 positive T cells from peripheral blood mononuclear cells (PBMCs);
(b) expanding the isolated naïve CD4 positive T cells;
(c) co-culturing the expanded naïve CD4 positive T cells with PBMCs and stimulating the expanded naïve CD4 positive T cells one or more times with the peptide; and
(d) detecting a signal corresponding to a level of one or more cytokines produced by the expanded naïve CD4 positive T cells.

77. The method of claim 76, wherein expanding the isolated naïve CD4 positive T cells leads to at least a 20-fold expansion of the isolated naïve CD4 positive T cells.

78. The method of claim 76, wherein expanding the isolated naïve CD4 positive T cells leads to at least a 30-fold expansion of the isolated naïve CD4 positive T cells.

79. The method of any one of claims 76 to 78, wherein the stimulating is performed in the presence of at least one helper cytokine.

80. The method of claim 79, wherein the at least one helper cytokine is selected from interleukin-2 (IL-2) and interleukin-7 (IL-7).

81. The method of claim 79, wherein the at least one helper cytokine is interleukin-2 (IL-2).

82. The method of any one of claims 76 to 81, wherein the stimulating is performed at least two times.

83. The method of any one of claims 76 to 81, wherein the stimulating is performed at least three times.

84. The method of any one of claims 76 to 81, wherein the stimulating is performed at least four times.

85. The method of any one of claims 76 to 84, wherein each of the stimulating steps is performed for a time period of about 1 day to about 3 days.

86. The method of any one of claims 76 to 84, wherein each of the stimulating steps is performed for a time period of about 3 days.

87. The method of any one of claims 76 to 86, wherein each of the stimulating steps comprises suspending the expanded naïve CD4 positive T cells in a fresh volume of media and stimulating the expanded naïve CD4 positive T cells with a fresh sample of the peptide.

88. The method of any one of claims 76 to 87, wherein each of the stimulating steps is performed in the presence of about 1 μg/mL to about 100 μg/mL of the peptide.

89. The method of any one of claims 76 to 87, wherein each of the stimulating steps is performed in the presence of about 10 g/mL to about 50 μg/ml of the peptide.

90. The method of any one of claims 76 to 87, wherein each of the stimulating steps is performed in the presence of about 40 g/mL of the peptide.

91. The method of any one of claims 76 to 90, wherein the co-culturing is performed with an excess of the expanded naïve CD4 positive T cells relative to the PBMCs.

92. The method of claim 91, wherein a ratio of the expanded naïve CD4 positive T cells to the PBMCs during the co-culturing ranges from about 2:1 to about 50:1.

93. The method of claim 91, wherein a ratio of the expanded naïve CD4 positive T cells to the PBMCs during the co-culturing is about 4:1.

94. The method of any one of claims 76 to 93, wherein expanding the isolated naïve CD4 positive T cells comprises exposing the isolated naïve CD4 positive T cells to magnetic beads coated with anti-CD3 antibodies and anti-CD28 antibodies.

95. The method of any one of claims 76 to 94, wherein detecting the signal corresponding to the level of the one or more cytokines comprises detecting the signal by an enzyme-linked immunoassay (ELISA) or enzyme-linked immunoassay (ELISpot).

96. The method of any one of claims 76 to 94, wherein detecting the signal corresponding to the level of one or more cytokines comprises detecting the signal by a flow cytometry-based assay.

97. The method of any one of claim 76 to 96, wherein the one or more cytokines detected in the detecting step include one or more anti-inflammatory cytokines.

98. The method of any one of claims 76 to 97, wherein the one or more cytokines detected in the detecting step include one or more pro-inflammatory cytokines.

99. The method of claim 97, wherein the one or more anti-inflammatory cytokines are selected from interleukin 10 (IL-10), interleukin 4 (IL-4), interleukin 13 (IL-13), interleukin 22 (IL-22), interleukin 6 (IL-6), and transforming growth factor beta (TGF-β).

100. The method of claim 97, wherein the one or more anti-inflammatory cytokines include interleukin 10 (IL-10).

101. The method of claim 98, wherein the one or more pro-inflammatory cytokines include interferon gamma (IFN-γ).

102. The method of any one of claims 76 to 101, wherein the signal corresponding to a level of one or more cytokines is detected in a media or in a cell.

103. A method of eliciting a release of one or more anti-inflammatory cytokines from an immune cell, wherein the method comprises contacting the immune cell with a peptide or an AIP of any one of claims 1 to 75.

104. A method of using a peptide or an AIP of any one of claims 1 to 75 as a positive control for eliciting release of at least one anti-inflammatory cytokine from a cell in an immune stimulating assay.

105. The method of claim 104, wherein the anti-inflammatory cytokine is selected from interleukin 10 (IL-10), interleukin 4 (IL-4), interleukin 13 (IL-13), interleukin 22 (IL-22), interleukin 6 (IL-6), transforming growth factor beta (TGF-β), and combinations thereof.

106. The method of claims 104 or 105, wherein the cell is an immune cell (e.g., a T cell).

107. The method of any one of claims 104 to 106, wherein the method comprises contacting the peptide or the AIP with the cell and detecting the release of the at least one anti-inflammatory cytokine.

108. A method of treating or preventing a disease or disorder in a subject in need thereof, wherein the method comprises administering a peptide or an AIP of any one of claims 1 to 75 to the subject.

109. The method of claim 108, wherein the disease or disorder is an inflammatory disease.

110. The method of claim 108, wherein the disease or disorder is an allergy or an allergic disease.

111. A pharmaceutical composition for treating or preventing a disease or disorder in a subject comprising a peptide or an AIP of any one of claims 1 to 75.

112. The pharmaceutical composition of claim 111, further comprising one or more pharmaceutically acceptable carriers or excipients.

113. The pharmaceutical composition of claims 111 or 112, wherein the disease or disorder is an inflammatory disease.

114. The pharmaceutical composition of claims 111 or 112, wherein the disease or disorder is an allergy or an allergic disease.

115. The pharmaceutical composition of claims 111 or 112, wherein the disease or disorder is associated with one or more anti-inflammatory cytokines.

116. The pharmaceutical composition of claim 115, wherein the disease or disorder is associated with the deficiency of the one or more anti-inflammatory cytokines.


SEQUENCE LISTING (45 sequences)

SEQ ID NO: 1 - protein, 12 aa, synthetic construct
KLAKRLLLSMNQ

SEQ ID NO: 2 - protein, 12 aa, synthetic construct
LALLAPKLLRRF

SEQ ID NO: 3 - protein, 13 aa, synthetic construct
FAVPAKQAKKTLT

SEQ ID NO: 4 - protein, 13 aa, synthetic construct
FAVWAKQAKKTLT

SEQ ID NO: 5 - protein, 13 aa, synthetic construct
FAVVAKQAKKTLT

SEQ ID NO: 6 - protein, 13 aa, synthetic construct
FAVMAKQAKKTLT

SEQ ID NO: 7 - protein, 13 aa, synthetic construct
FAVIAKQAKKTLT

SEQ ID NO: 8 - protein, 13 aa, synthetic construct
FAVFAKQAKKTLT

SEQ ID NO: 9 - protein, 13 aa, synthetic construct
FAVPAYQAKKTLT

SEQ ID NO: 10 - protein, 13 aa, synthetic construct
FAVPAWQAKKTLT

SEQ ID NO: 11 - protein, 13 aa, synthetic construct
FAVPAFQAKKTLT

SEQ ID NO: 12 - protein, 13 aa, synthetic construct
FAVWAYQAKKTLT

SEQ ID NO: 13 - protein, 13 aa, synthetic construct
FAVWAWQAKKTLT

SEQ ID NO: 14 - protein, 13 aa, synthetic construct
FAVWAFQAKKTLT

SEQ ID NO: 15 - protein, 13 aa, synthetic construct
FAVVAYQAKKTLT

SEQ ID NO: 16 - protein, 13 aa, synthetic construct
FAVVAWQAKKTLT

SEQ ID NO: 17 - protein, 13 aa, synthetic construct
FAVVAFQAKKTLT

SEQ ID NO: 18 - protein, 13 aa, synthetic construct
FAVMAYQAKKTLT

SEQ ID NO: 19 - protein, 13 aa, synthetic construct
FAVMAWQAKKTLT

SEQ ID NO: 20 - protein, 13 aa, synthetic construct
FAVMAFQAKKTLT

SEQ ID NO: 21 - protein, 13 aa, synthetic construct
FAVIAYQAKKTLT

SEQ ID NO: 22 - protein, 13 aa, synthetic construct
FAVIAWQAKKTLT

SEQ ID NO: 23 - protein, 13 aa, synthetic construct
FAVIAFQAKKTLT

SEQ ID NO: 24 - protein, 13 aa, synthetic construct
FAVFAYQAKKTLT

SEQ ID NO: 25 - protein, 13 aa, synthetic construct
FAVFAWQAKKTLT

SEQ ID NO: 26 - protein, 13 aa, synthetic construct
FAVFAFQAKKTLT

SEQ ID NO: 27 - protein, 12 aa, synthetic construct
LYLLAPKLLRRF

SEQ ID NO: 28 - protein, 12 aa, synthetic construct
LWLLAPKLLRRF

SEQ ID NO: 29 - protein, 12 aa, synthetic construct
LFLLAPKLLRRF

SEQ ID NO: 30 - protein, 12 aa, synthetic construct
LALLAPKLLRRK

SEQ ID NO: 31 - protein, 12 aa, synthetic construct
LALLAPKLLRRE

SEQ ID NO: 32 - protein, 12 aa, synthetic construct
LALLAPKLLRRD

SEQ ID NO: 33 - protein, 12 aa, synthetic construct
LALLAPKLLRRA

SEQ ID NO: 34 - protein, 12 aa, synthetic construct
LYLLAPKLLRRK

SEQ ID NO: 35 - protein, 12 aa, synthetic construct
LYLLAPKLLRRE

SEQ ID NO: 36 - protein, 12 aa, synthetic construct
LYLLAPKLLRRD

SEQ ID NO: 37 - protein, 12 aa, synthetic construct
LYLLAPKLLRRA

SEQ ID NO: 38 - protein, 12 aa, synthetic construct
LWLLAPKLLRRK

SEQ ID NO: 39 - protein, 12 aa, synthetic construct
LWLLAPKLLRRE

SEQ ID NO: 40 - protein, 12 aa, synthetic construct
LWLLAPKLLRRD

SEQ ID NO: 41 - protein, 12 aa, synthetic construct
LWLLAPKLLRRA

SEQ ID NO: 42 - protein, 12 aa, synthetic construct
LFLLAPKLLRRK

SEQ ID NO: 43 - protein, 12 aa, synthetic construct
LFLLAPKLLRRE

SEQ ID NO: 44 - protein, 12 aa, synthetic construct
LFLLAPKLLRRD

SEQ ID NO: 45 - protein, 12 aa, synthetic construct
LFLLAPKLLRRA